(19) United States
(12) Patent Application Publication
(10) Pub. No.: US 2016/0310020 A1
(43) Pub. Date: Oct. 27, 2016

(54) SYSTEM FOR DETECTION OF FLUID PRESSURE USING A PRESSURE SENSING CAPACITIVE SENSOR

(71) Applicant: Guided Interventions Inc., Elyria, OH (US)

(72) Inventors: Reinhard WARNKING, Setauket, NY (US); Joerg SCHULZE-CLEWING, Cameron Park, CA (US); David DiPAOLA, North Potomac, MD (US); Dong lk SHIN, Poway, CA (US); Matthew J. POLLMAN, San Francisco, CA (US)

(21) Appl. No.: 15/104,896

(22) PCT Filed: Sep. 11, 2014
(86) PCT No.: PCT/US2014/055184

§ 371 Date: Jun. 15, 2016

Related U.S. Application Data
(60) Provisional application No. 62/027,088, filed on Jul. 21, 2014.
(60) Provisional application No. 61/920,291, filed on Dec. 23, 2013.

(51) Int. Cl. A61B 5/0215; A61B 5/00

(52) U.S. Cl. CPC A61B 5/0215; A61B 5/7257; A61B 5/6852; A61B 5/6851; A61B 5/0022

(57) ABSTRACT

A system having a catheter having a distal pressure sensitive capacitive element providing an impedance and phase shift which varies responsive to the amount of pressure from blood external the catheter, a detector having electronics for determining the impedance and/or phase shift. This impedance and or phase shift corresponds to the pressure of the blood about the distal end of the catheter. When the catheter is inserted into a patient's body, the impedance or phase shift is detected quasi wirelessly without special signal communication means like optical fibers or electrical wires from outside the patient's body utilizing the patient as a ground path and the catheter shaft as an electrical conductor.

### FIELD OF THE INVENTION

The present invention is related to a system (apparatus and method) for detection of fluid pressure, such as blood pressure in a blood vessel. The invention is directed in part to a system having a catheter with a sensor for sensing pressure of the blood, and a method using same for monitoring the blood pressure sensed.

### BACKGROUND OF THE INVENTION

Interventional cardiologists rely on guide wires to reach the treatment site inside a blood vessel, such as the coronary arteries. Instead of utilizing the guide wire as a strictly mechanical- or guiding-tool, pressure and flow wires are being promoted as a dual function guide wires, providing mechanical guidance and hemodynamic information at the same time. Based on the results of the FAME and DEFER studies (see Pijls et al. “Percutaneous Coronary Intervention of Functionally Nonsignificant Stenosis 5 Year Follow Up of the DEFER Study”. J Am Coll Cardiol 2007 vol 49 (21) pp 2105-2111), FFR (Fractional Flow Reserve) measurements are becoming popular and in several countries reimbursed. Currently there are 2 types of pressure wires commercially available: Radi (acquired by STJ) and Volcano. Both guide wires use an IC pressure sensor (strain gage type) connected through a push on handle at the proximal wire end. In case of the Radi guide wire, the connector handle wirelessly transmits pressure values to a display system. This is an improvement over a cable connection, however, still very cumbersome, since for every catheter insertion the connector handle needs to be disconnected from the proximal wire end before the catheter can be advanced over the wire. Also, these FFR wires are complex in structure since special auxiliary signal communication lines need to be routed along the guide wire. Signal communication means consist of 3 electrical wires in case of piezo resistive sensors or an optical fiber in case of fiber optical sensors. This integration of signal communication transmission paths greatly degrades guide wire handling and mechanical guide wire performance. In particular steer-ability, torque-ability and push-ability are inferior with FFR wires compared to standard guide wires. One approach to overcome the guide wire degradation is to utilize a pressure sensing catheter over a standard guidewire. The challenge is to keep the catheter dimensions to a minimum in order to not impact flow through the lesion site by reducing the blood flow cross section further through the catheter inside the lesion. Unless catheter dimensions can be kept to a minimum, comparable to guide-wire dimensions, the flow reduction through the catheter needs to be compensated for as described in US Patent Application Publication No. 20140066765; U.S. Pat. No. 8,696,584B2 and International Patent Application Publication NO. WO 201425255A1.

### SUMMARY OF THE INVENTION

The present invention aims to optimize and facilitate in vivo fluid pressure measurements, for example, by performing FFR measurements with a small catheter advanced over a standard guide wire. More particularly, the present invention contemplates obtaining in vivo fluid pressure measurements by utilizing a catheter with just one electrical contact to be made to the measurement system. Therefore the catheter structure itself can be utilized to serve as an electrical conductor and no special or dedicated signal communication lines need to be integrated into the catheter, keeping the dimensions of the catheter to a minimum while optimizing handling characteristics. It is to be noted that the present invention also contemplates blood pressure measurement systems where a catheter is omitted. Where a catheter is included in the system, the sensor is incorporated into the wall of the catheter. Where a solid wire is provided which extends parallel to a steering guide wire, the sensor may be integrated into the solid wire. In one embodiment, the present invention is directed to a FFR catheter system utilizing a standard or off-the-shelf guide wire. The catheter has a distal portion with a capacitive sensor. The capacitive sensor is either an electrolytic sensor (a capacitor with an electrolyte reservoir between capacitor plates) or a MEMS sensor as described in International Patent Application No. PCT/US2014/023358 filed Mar. 11, 2014 (Exhibit B). Both sensor types provide very high capacitive values allowing construction of the FFR catheter without having to integrate special signal communication means like electrical wires or optical fibers as described in PCT/CA2010/000396. This enables to minimize the catheter dimensions and to minimize the flow reducing impact of the catheter inside the lesion and thereby increase the accuracy of the FFR measurement. Also, catheter handling is greatly improved since no signal communication means like electrical wires or optical fibers need to be integrated into the catheter shaft, as described in Application No. PCT/CA2010/000396, which would compromise mechanical parameters like torque-ability, push-ability and bend-ability. Signal transmission pursuant to the present invention is accomplished by utilizing the patient body as one conductor or transmission path and the catheter shaft as the second conductor or transmission path. The high capacitive value of the sensor in the nF range allows blood pressure dependent sensor values to be sensed despite significant leakage-capacitances and -impedances inside the patient body.

### BRIEF DESCRIPTION OF THE DRAWINGS

The foregoing objects, features and advantages of the invention will become more apparent from a reading of the following description in connection with the accompanying drawings, in which:

FIG. 1 is partially a schematic perspective view and partially a block diagram of a wireless pressure sensing system.

FIG. 2 is partially a schematic cross-sectional view and partially a circuit diagram of a distal portion of a guide wire in an artery of a patient, in conformity with the present invention.

FIG. 3 is a circuit diagram of an external circuit of the wireless pressure sensing system of FIG. 1.

FIGS. 4A and 4B are two perspective views illustrating the operation of two resonance circuits which may represent a sensor circuit and a detector circuit, where FIG. 4A shows the sensor circuit and the detector circuit in resonance and accordingly a high current on an oscilloscope, and FIG. 4B shows the sensor circuit detuned with an additional capacitor which reduces the current into the detector circuit and therewith the current on the oscilloscope.

FIG. 5 is a schematic side elevational view of a distal end portion of a guide wire, showing a coil implementing a floppy tip and utilized as the inductor of a pressure sensing resonance circuit and further showing the position of a variable-capacitance capacitor completing the resonance circuit.

FIG. 6 is a circuit diagram showing a 2 contact version of a pressure-sensing guide wire system.

FIG. 7 is a schematic side elevational view of the 2 contact version of the pressure-sensing guide wire, demonstrating how a core wire and a hypotube are utilized as electrical conductors.

FIG. 8 is a series of three different resonance curves representing different capacitance values and concomitantly different pressure values.

FIG. 9 is essentially a circuit diagram of a sheath contact version of a pressure sensing guide wire system.

FIG. 10 is a schematic side elevational view of the sheath-contact pressure wire depicted as a circuit diagram in FIG. 9.

FIG. 11A is a diagram of a sensor resonance circuit in a distal portion of a guide wire, where the circuit includes a fixed value capacitor and a pressure sensitive inductor.

FIG. 11B is a diagram similar to FIG. 11A, showing the inductor with a shorter length owing to contraction in response to an increase in surrounding pressure.

FIG. 12A is a diagram of another resonance circuit in the distal portion of a guide wire with a fixed value capacitor and a pressure sensitive inductor.

FIG. 12B is a diagram similar to FIG. 12A, showing the inductor with a shorter length owing to contraction in response to an increase in surrounding pressure.

FIG. 13A is a diagram of yet another resonance circuit in the distal portion of a guide wire with a fixed value capacitor and a pressure sensitive inductor by virtue of a shiftable ferromagnetic inductor core.

FIG. 13B is a diagram similar to FIG. 12A, showing the core inserted to a greater extent inside an inductor coil in response to an increase in surrounding pressure.

FIG. 14 is partially a schematic perspective view and partially a block diagram of another wireless or practically wireless pressure sensing system, showing an external detection unit connected to an external coil located at a distal end of an insertion sheath.

FIG. 15 is partially a schematic perspective view and partially a circuit diagram of another contactless pressure sensing system, where an external detector includes a radio transmitter.

FIG. 16 is a schematic diagram of the contactless configuration of FIG. 15, showing how a proximal guide wire end acts as an opposite (receiver) antenna.

FIG. 17 is a schematic diagram of another contactless configuration wherein a detector and guide wire are coupled capacitively through an insertion sheath.

FIG. 18 is a schematic perspective view of an adhesive patch carrying a resonance circuit inductor.

FIG. 19 is a schematic side elevational view of an inductor or coil having a number of active windings that varies in accordance with external fluid pressure.

FIG. 20 is essentially a circuit diagram of a pressure sensing guide wire system, with a ceramic resonator and a capacitive sensor, schematically showing deployment thereof in a human patient for purposes of measuring blood pressure.

FIG. 21 is a diagram of a tuning-fork-type MEMS resonator device for fluid pressure measurement.

FIG. 22 is a circuit diagram similar to FIG. 20 but incorporating the tuning-fork-type MEMS resonator device of FIG. 21.

FIG. 23 is a schematic side elevational view, partly in cross-section, of a multi-layer ceramic capacitor utilizable in an LC pressure measurement device as disclosed herein with reference to FIGS. 1-19.

FIG. 24 is a block diagram of an electronic signal processing circuit as a component part of a pressure sensing guide wire system.

FIG. 25 is a block diagram of another electronic signal processing circuit as a component part of a pressure sensing guide wire system.

FIG. 26 is a block diagram of a directional coupler showing connections for an electronic amplitude monitoring circuit for a resonator-incorporating resonance circuit.

FIG. 27 is a block diagram of a pressure-measuring resonance circuit for operating in the time domain.

FIG. 28 is a drawing showing another embodiment of a pressure-sensing device, with components mounted inside a 14/1000 of an inch guide wire.

FIG. 29 is a diagram of the distal portion of the guide wire of FIG. 28 showing a different configuration for the sensor.

FIG. 30 is a schematic diagram of the distal portion of the guide wire of FIG. 28 showing another sensor configuration.

FIG. 31 is essentially a schematic side elevation diagram showing the distal end of a guide catheter inside the aorta with a distal guide wire extending into a coronary vessel.

FIG. 32 is a schematic perspective view broken away to show layers of a sheath or guide catheter and an inserted guide wire.

FIG. 33 is a schematic elevational view, partially broken away, of a proximal end of a sheath or guide catheter portion (hub), showing an external brush contact.

FIG. 34 is a schematic elevational view of a proximal end of a sheath or guide catheter portion (hub), showing a guide wire inserted and a wire torquer attached to the proximal end of the guide wire, electrically connecting the guide wire with an FFR system.

FIG. 35 is an electrical diagram of a resonance circuit of FIG. 31 connected to a phase detection system through the core wire of the guide wire and with ground electrodes connected through the blood stream.

FIG. 36 is a schematic elevational view of a proximal end of a sheath or guide catheter portion (hub), showing a circuit connection through a tubular ground electrode connecting the FFR system to the patient's blood stream, which does not require sheath modifications.

FIG. 37 is a diagram of a guide wire with a capacitive sensor inside a patient and electrical connections established through a sheath contact and ground electrode.

FIG. 38 is a schematic perspective view, partially broken away, of the guide wire of FIG. 37, showing the position of the capacitive sensor in a floppy tip section of the guide wire.

FIG. 39 shows a computer display screen with in vivo phase measurement of the parasitic wire/body capacitance.

FIG. 40 shows a display screen recording in vivo phase measurement of the parasitic capacitance variations with breathing and heart cycle.

FIG. 41 is a table of key findings from in vivo measurements of parasitic impedance parameters.

FIG. 42 shows a display screen with an amplitude measurement of bloodstream impedance changes with cardiac and breathing cycles.

FIGS. 43 and 44 are perspective views of a capacitive sensor utilizable in a pressure sensing system, showing top and bottom sides, with first and second capacitors in parallel and solder bumps as one form of electrical interconnect.

FIG. 45 is a schematic side elevational view, on an enlarged scale, which shows the capacitive sensor of FIGS. 43 and 44 connected to wires which make the electrical connection to a distal and proximal guide wire portion.

FIG. 46 shows a cross-section view of 1 of 2 capacitors of the capacitive sensor of FIGS. 43-44, depicting dielectric and conductive layers building up the MEMS capacitive sensor.

FIG. 47A is, in pertinent part, an overall electrical block diagram of a system of the present invention with the FFR catheter shown in an OTW (Over The Wire) implementation inserted inside a patient.

FIG. 47B is a simplified partial schematic perspective view of the OTW catheter assembly of FIG. 47A.

FIG. 48 is a schematic partial longitudinal cross-sectional view of a catheter in accordance with the present invention, showing an electrolyte sensor type mounted in an OTW FFR catheter assembly.

FIGS. 49A and 49B are schematic isometric top and bottom views of a MEMS sensor utilizable in a catheter or in vivo pressure sensing assembly in accordance with the present invention.

FIG. 50 is a schematic perspective view of a pressure-sensing device or assembly incorporating an embodiment of the present invention, where a sensor-carrying elongate flexible wire member is inserted into a patient's vascular system in parallel to a guide wire (rapid exchange version).

FIG. 51 shows a cross section through the MEMS sensor of FIGS. 49A and 49B, utilizable in the pressure-sensing device or assembly of FIG. 50.

FIGS. 52A and 52B are block diagrams, showing respective variants of a detector circuit which may be used in a fluid pressure sensing system in accordance with the present invention.

FIG. 53 is a schematic perspective view of a MEMS sensor mounted in a rapid exchange FFR catheter assembly in accordance with the present invention.

### DETAILED DESCRIPTION

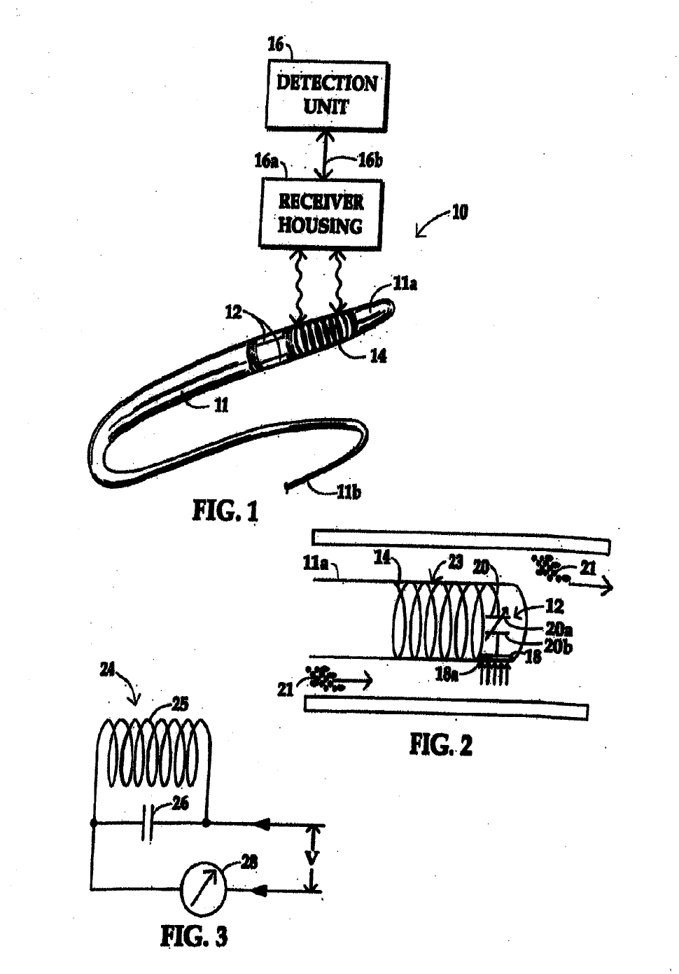

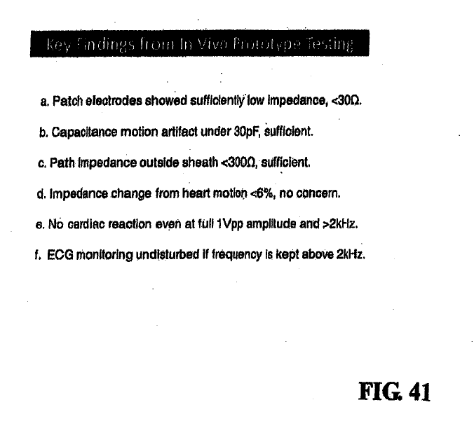

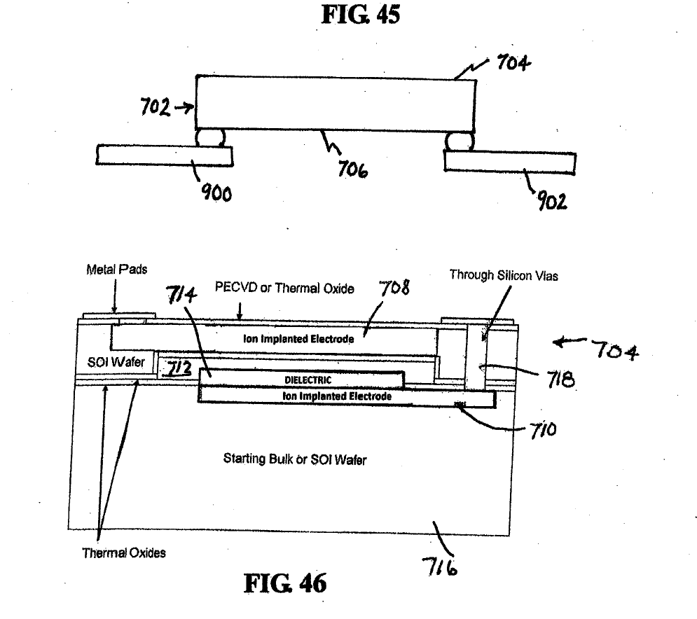

FIGS. 1-46 and the associated description thereof hereinafter are directed to a pressure sensing system where a sensor is incorporated in an interventional medical guide wire. Pursuant to FIGS. 47AS through 53, a pressure sensor may be provided in a catheter, imbedded in the catheter wall, or in an elongate shaft that extends in parallel to an introducing guide wire.

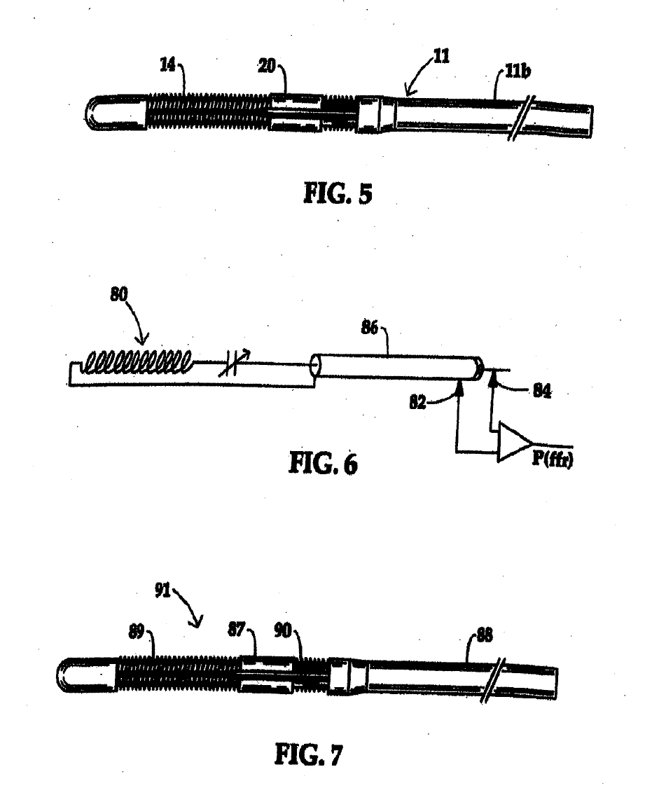

As illustrated in FIG. 1, a pressure sensing guide wire system 10 comprises a guide wire 11 having a sensor 12 and coil 14 at its distal end portion 11a. FIG. 5 shows the mechanical arrangement of a floppy tip coil 14 and a capacitive sensor 20 forming a pressure sensing resonance circuit. The guide wire 11 may be inserted into the cardiovascular system of a patient. Small flexible devices, called catheters, may be guided over the guide wire 11 inserted through blood vessels and vascular structures of the patient, such as to the site of a damaged or diseased blood vessel, as typically performed in interventional cardiology. A detection unit 16 has a receiver housing 16a disposable external of the patient's body in the vicinity of the resonance circuit consisting of sensor 12 and coil 14. In a typical embodiment, receiver housing 16a carries an inductor 25 (FIG. 3) that may take the form of a flat coil, particularly a printed coil, attachable to the patient's side roughly at the location of the heart in case of coronary artery interventions. Such printed circuit coils are preferably disposable. The receiver housing 16a may be in contact with the patient's skin surface or introduced within the patient. Information from the sensor 12 is wirelessly detected by the receiver (detection resonance circuit 24, see FIG. 3) through the human body (soft or hard tissue). The body 11b of the guide wire 11, which is integrated with the sensor 12 and the coil 14, may be a typical guide wire used in interventional cardiology or interventional radiology (i.e., composed of non-corrosive biocompatible material(s)) and of a diameter and sufficiently flexibly and bendable to pass through blood vessel(s) or vascular structure(s) to a surgical or diagnostic target site in the patient (see also FIG. 5). The sensor 12 and detection unit 16 provide wireless detection of a physical variable, in particular blood pressure at such site, thus eliminating the need for a mechanical and electrical connection between the sensor and external detection equipment of the prior art.

FIG. 2 shows the distal portion 11a of the guide wire 11 in more detail. The distal portion 11a is a cone integrated to the body 11b at the distal end of the guide wire. The sensor 12 comprises a pressure sensitive element 18 mounted in the guide wire to detect the blood pressure surrounding the wire 11, and further comprises a variable capacitor 20, which is referred to herein as a pressure sensitive capacitive element. Pressure sensitive element 18 is connected to or part of a variable capacitor 20 whose capacitance value varies with amount of pressure upon element 18 from the blood about the distal portion 11a. The pressure sensitive element 18 has an outer surface 18a exposed to blood 21 about the distal wire portion 11a, and may be biased, such as by a spring, away from capacitor 20. Increased or decreased pressure upon the outer surface 18a moves the pressure sensitive element towards or away, respectively, from the capacitor, the change in distance resulting in a change in the capacitive value of capacitor 20 and hence the resonance frequency of the resonance circuit 23 (FIG. 2) consisting of capacitor 20 connected to coil 14. More specifically, capacitor 20 may include a first plate element 20a and a second plate element 20b, where the latter is movably mounted relative to plate element 20a and guide wire 11 and coupled or entrained to pressure sensitive element 18 so that motion of the pressure sensitive element causes a change in the distance between plate 20b and 20a. Other capacitive pressure sensors may also be used, such as described for example in Sensors and Actuators A: Physical Vol. 73, Issues 1-2, 9 Mar. 1999, Pages 58-67 or as shown in FIGS. 29, 30 and 46, 51 hereof.

The position of the coil 14 and sensor 12 in the distal portion 11a of the guide wire may either be as shown in FIG. 1, in which the coil is more distal than the sensor 12, or vice versa, as shown in FIG. 2.

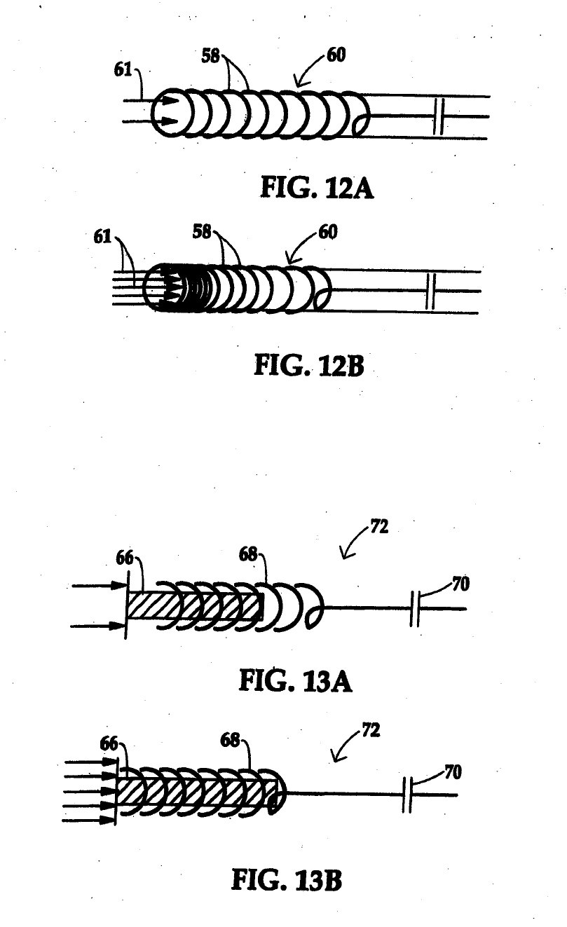

The coil 14 provides an inductance which may utilize the coil tip (or sections thereof) at the distal end of the guide wire, often referred to as the floppy tip. This inductor 14 and pressure sensitive capacitor 20 form a resonance circuit 23 with a resonance frequency varying with blood pressure fluctuations. In other embodiments, the capacitor can be of fixed value while the inductance of the coil changes according to the surrounding blood pressure. This can be accomplished by changing the length of a coil 56 or 60 according to surrounding blood pressure as shown in FIGS. 11A and 11B or FIGS. 12A and 12B. In the approach of FIGS. 12A and 12B, windings 58 of coil 60 are pressed in a longitudinal or axial direction of the guide wire in response to fluid pressure 61 exerted in that direction. Inductance changes can also be related to the surrounding blood pressure by changing the number of active windings of a coil or by changing the position of a ferromagnetic core 66 inside the coil as shown in FIGS. 13A and 13B.

In the embodiment of the wireless pressure sensing guide wire system of FIGS. 1-3, an external or extracorporeal electro-magnetic field is created in response to an applied voltage by an external resonance (or detector) circuit 24 of the detection unit 16, comprising a capacitor 26 and an inductor (or coil) 25 as shown in FIG. 3. When both resonance circuits 23 and 24 are tuned to the same resonance frequency, a maximum energy transfer will take place from the external circuit 24 to the internal circuit 23, which is mounted inside the guide wire 11. Detuning of the circuit 23 through capacitance changes (caused by blood pressure variations) will vary the amount of transmitted energy to the external circuit 24. By recording the changes of transmitted energy, a blood pressure recording is provided, as via a current sensor 28. Thus, pressure values are detected without making an electrical connection by wire at the proximal guide-wire end or by switching the detector unit 16 into a receive-only mode relying on very weak signals being emitted from a free oscillation of the sensor circuit 12 after the power to the detector circuit 16 has been cut, as described in U.S. Pat. No. 6,517,481.

The detection circuit 24 may be disposed in housing 16a and electronically connected (e.g., via wires 16b) to the detection unit 16 which supplies power and varies the frequency of resonance circuit 24 in the operative frequency range of circuits 23 and 24, and a change in power/current monitor 28 detects the resonance frequency when circuits 23 and 24 are in resonance.

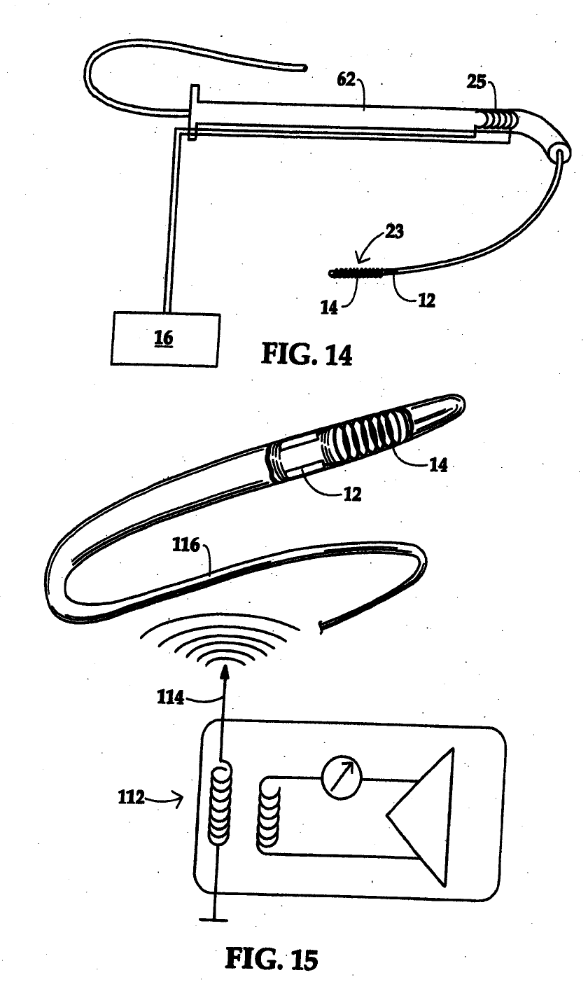

Optionally, in order to improve the coupling between sensor circuit 23 and detector circuit 24, the coil 25 of detector circuit 24 may be located in an insertion sheath 62 rather than housing 16a as shown in FIG. 14. During use of the pressure sensing guide wire system of FIG. 14, the sheath 62 may be located inside the aorta of the patient and the distal sheath end at the aortic arch and all devices (guide wire 11, balloon catheters etc) are advanced through the sheath. This has the advantage of better coupling between sensor circuit 23 and detector circuit 24. The guide wire 11 may contain a core wire which may be fabricated out of a ferromagnetic material to even further improve coupling, since sensor coil 14 and detector coil 25 surround the same ferromagnetic core as shown in FIG. 14.

Only one LC circuit 23 is provided in the guide wire 11: an inductance L consisting of wire windings or coil 14 in the floppy tip 11a of the wire 11 and a capacitor 20 which changes capacitance C with blood pressure.

The inductance L of a distal pressure-sensing coil or inductor may be varied by moving, in response to blood pressure, a ferromagnetic core member 66 inside a guide wire coil 68 which is connected together with a fixed-value capacitor 70 in a resonance circuit 72, as shown in FIGS. 13A and 13B. Alternatively, the resonance frequency of an LC circuit may be varied in accordance with blood pressure by changing the number of active windings of a variable-inductance coil. This change in the number of active windings may be accomplished by shifting a winding-contact element and the coil relative to one another. Pursuant to another approach, depicted in FIGS. 12A and 12B, the inductance is adjusted by compressing, through blood pressure, the coil 60 as shown in FIGS. 12A and 12B. Changing the length of the coil 60, in response to a blood pressure-induced axial force serves to vary the inductance of the coil. In another embodiment, shown in FIGS. 11A and 11B, a membrane 74 surrounding the coil 56 is compressed in a transverse or radial direction by the surrounding blood pressure 75. With windings 78 of coil 56 movably mounted relative to the guide wire and with the membrane 74 connected to the windings, the inward distortion of the membrane 74 causes the windings 78 to move laterally towards one another, in the longitudinal direction of the guide wire, thus modifying the active length of the coil 56 and varying the inductance proportional to blood pressure changes.

In system 10 of the present invention contact-less detection of a remote sensor is accomplished by detecting the resonant frequency of the sensor circuit 23 while the external detector circuit 24 is being powered up. The detection operation works as follows: the external high frequency oscillator sweeps across a frequency band. An electromagnetic field of different frequencies is generated while the power consumption of the external high-frequency oscillator is being monitored. The sensing LC circuit 23 absorbs a portion of the RF power of external high frequency oscillator mainly at its resonant frequency. The power, with which the external oscillator is supplied, will exhibit a change when the external circuit 24 and the sensing circuit 23 are in resonance. This change in power consumption of the external high frequency oscillator represents the resonance frequency of the LC sensor 12 which in turn is indicative of the blood pressure.

The detection unit 16 may have electronics for detecting when the power change occurs and displaying the corresponding blood pressure reading on a display. Such electronics may have a programmed controller or microprocessor (or other logic device), which calculates (or lookups up in a table in a memory) the corresponding blood pressure for the detected resonance frequency for output to the display. The relationship of resonance frequency to blood pressure may be in accordance with an equation, or calibrated with circuits 23 and 24 to provide a curve or look-up-table relating frequency to blood pressure stored in memory of the electronics for later use. See for example, see monitoring material properties in: Butler; Sensors and Actuators A 102 (2002)61-66. The blood pressure monitoring process may be done periodically during interventional procedures or as needed to classify the hemodynamic significance of a lesion, so that the blood pressure about the site of intervention can be accurately measured.

Detection unit 16 is configured for detecting a change in blood pressure by detecting an absorption of less electromagnetic energy by resonance circuit 23 in response to the change in the inductance or capacitance of the pressure-sensitive LC circuit element. Detection unit may be programmed to calculate, or look up in a table, the pressure corresponding to the amount of reduction of energy absorption. Alternatively, detection unit 16 may induce detector circuit 24 to scan through a range of frequencies about the former resonance frequency, thereby picking up or detecting a new resonance frequency. Detection unit 16 may then report the new blood pressure associated with the newly detected resonance frequency.

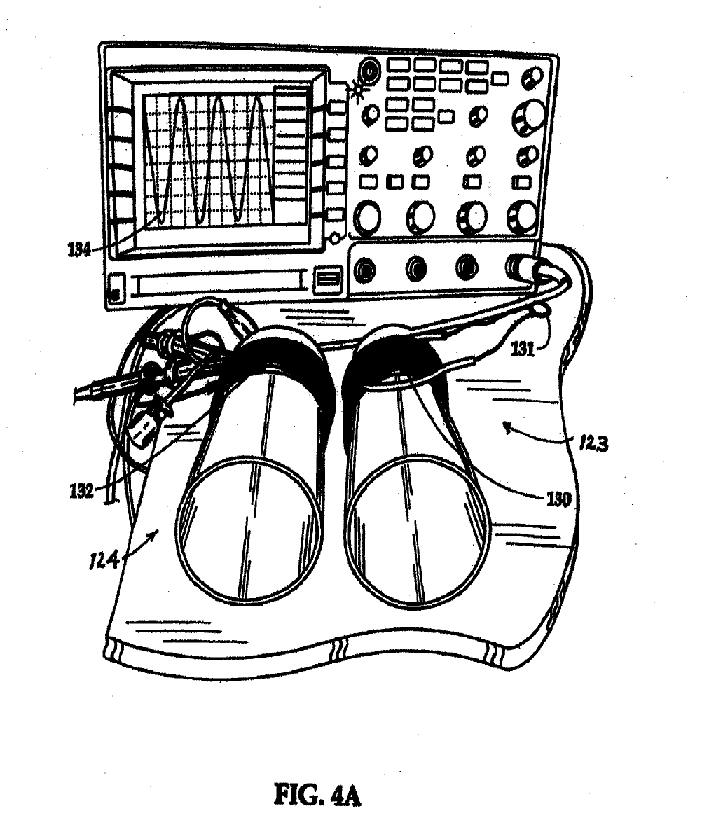

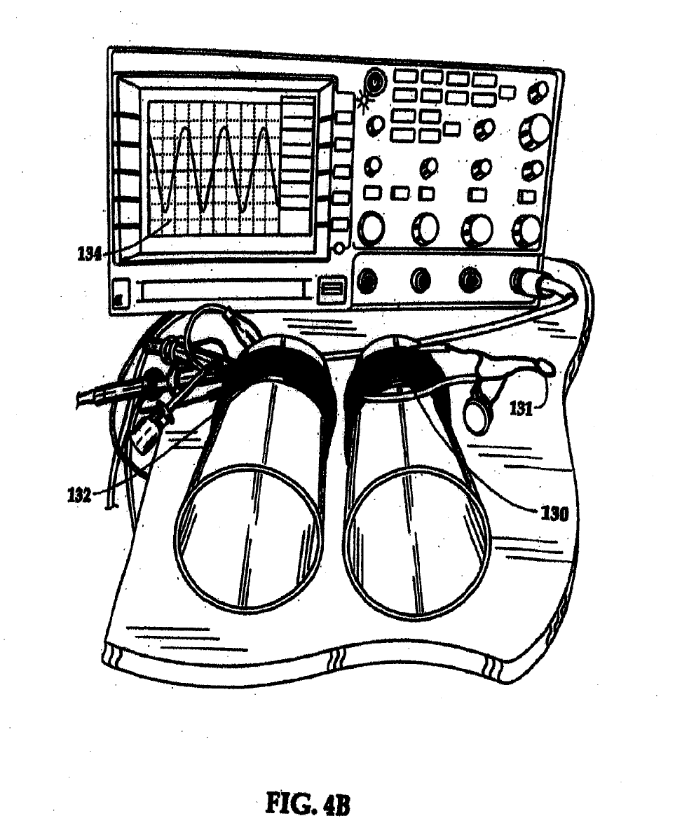

FIGS. 4A and 4B are two perspective views illustrating resonance between two resonance circuits illustrating the operation of the present invention providing a sensor circuit 123, which corresponds to and functions the same way as sensor circuit 23, and a detector circuit 124, which corresponds to and functions the same way as detector circuit 24 of system 10. The sensor circuit 123 for illustrative purposes is not shown in the desired form and configuration described earlier. The detector circuit 124 may also be in a different form than shown. In each FIG., the right circuit illustrates the sensor circuit 123 having a coil 130 connected to capacitor 131, the left circuit illustrates the detector circuit 124 having a coil 132 connected to a capacitor (not shown), and the oscilloscope's leads are connected to the detector circuit. FIG. 4A shows the sensor circuit and the detector circuit in resonance and accordingly a high current on the oscilloscope' screen 134 at this frequency. A frequency oscillator (not shown) when such resonance circuits are in the desired form and configuration coupled to the detector circuit was varied until the high current was observed on the oscilloscope (i.e., from a change in power consumption of the detector circuit 24 when the two circuits illustrated are in resonance). To illustrate a pressure change (and hence capacitance), FIG. 4B illustrates the sensor circuit detuned with an additional capacitor 132 connected to capacitor 131, which reduces the current in the detector circuit and hence the observed current is now lower on the oscilloscope's screen 134. The frequency oscillating the detector circuit is now at the different frequency than the new resonance frequency of the sensor circuit due to the combined capacitance of capacitors 132 and 131 in the LC circuit 23 with coil 130.

From the foregoing description, it will be apparent that there have been provided a wireless pressure sensing guide wire and detector. Variations and modifications in the herein described apparatus, method, and system in accordance with the invention will undoubtedly suggest themselves to those skilled in the art.

FIG. 6 is an electric circuit diagram showing the structure of the 2 contact pressure wire version. A resonance sensing circuit 80 at the distal wire end is identical to the one described above for the wireless version. Instead of wirelessly determining the change of the resonance frequency, two contacts 82 and 84 at the proximal wire end 86 are utilized.

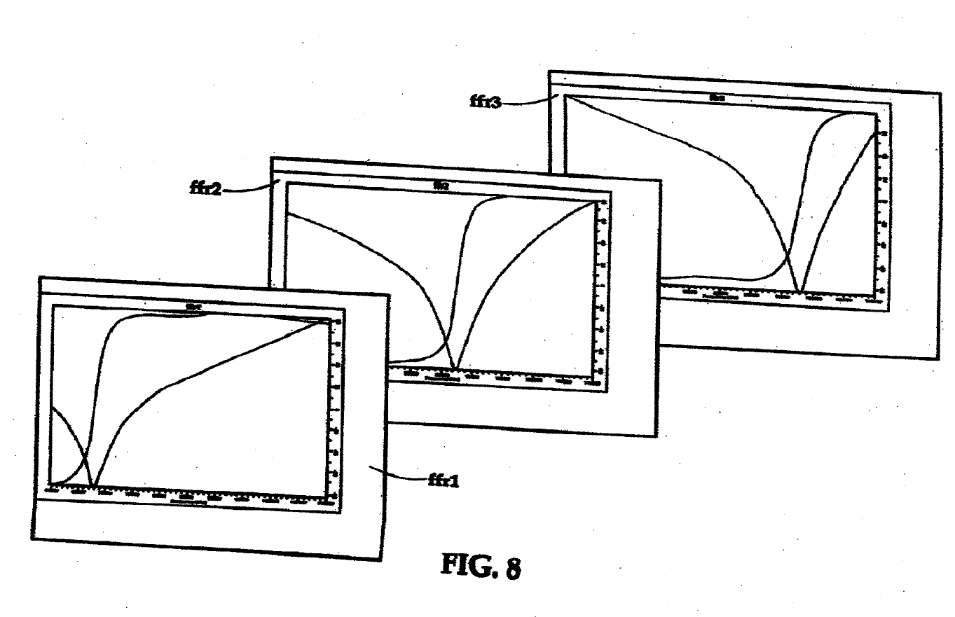

FIG. 8 demonstrates the change in resonance frequencies for capacitive values of about 13 pF in screen ffr1, about 8 pF in screen ffr2 and about 7 pF in screen ffr3. A change of about 5 to 6 pF represents the physiological pressure range in this experiment and allows for unmistaken detection of the blood pressure values. FIG. 7 shows typical guide wire components utilized as electrical conductors to avoid having to integrate additional electrical wires into the guide wire structure, which negatively affects wire handling. The compromised wire handling of the commercially available pressure sensing guide wires represents a significant barrier towards widespread use of pressure sensing guide wires. As FIG. 7 demonstrates, the wire handling can be equal to non-pressure sensing guide wires by requiring only 2 electrical conductors in the coaxial form of the standard wire components of hypotube 88 and core wire 90). Core wire 90 is connected to a capacitor 87 and inductor or coil 89 of an LC pressure-sensing circuit 91.

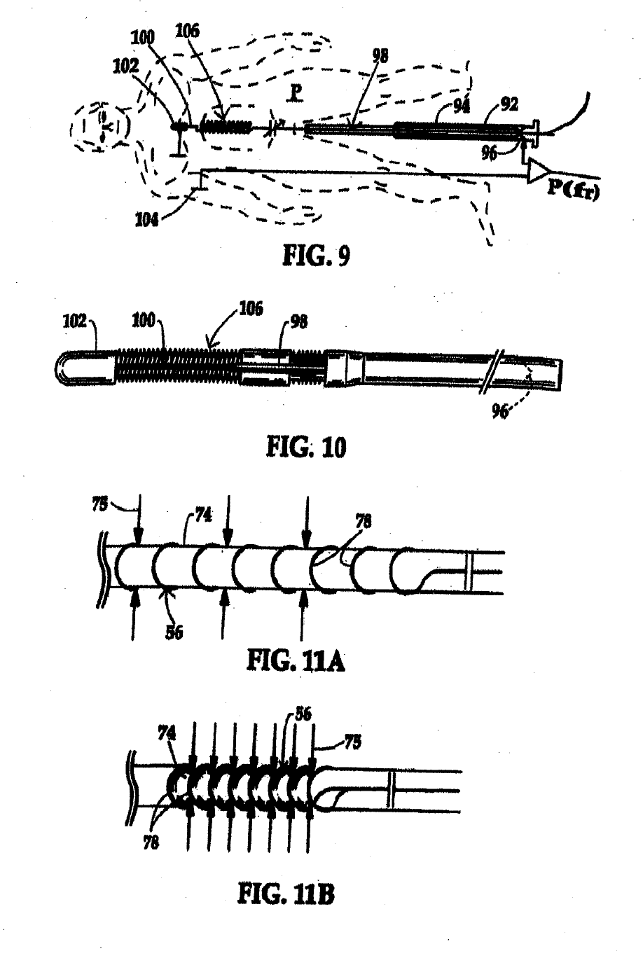

FIG. 9 shows an alternative configuration which to the user appears wireless since a proximal guide wire end 92 does not need to be connected with a connector handle. Instead a sheath 94, which is part of any interventional procedure, contains a brush contact 96 to connect to the proximal end 92 of the guide wire 98, while a distal end 100 of the wire is in electrical contact via an electrode 102 with the patient P, who in turn is connected to ground potential through a ground electrode 104. This grounding technique is widely utilized in RF ablation procedures with a typical impedance of about 100 Ohms from RF electrode to ground. As can be seen in FIG. 8, the resonance frequencies, for the pressure wire configurations described here, are in the 10^{th} of MHz range (vs. KHz range for RF ablations), which reduces the serial impedance to ground to negligible values since the mostly capacitive impedance of the patient body is proportional to 1/f. An LC resonance circuit 106 at the distal wire end 100 is connected through electrode 102 at the distal tip of the wire 98 to the patient P who is connected to ground potential through the ground electrode 104. The other end of the resonance circuit 106 is connected to the proximal wire body 98, either the hypotube and or core wire or a solid proximal wire portion. The proximal end portion 92 of the wire 98 is not insulated in order to make contact with the contact brush 96 within the sheath as shown in FIG. 10. This has the same advantage as in the two contact version that wire handling is not compromised since standard wire components (hypotube and or core wire) are utilized as electrical conductors avoiding the insertion of additional electrical wires or signal communication means.

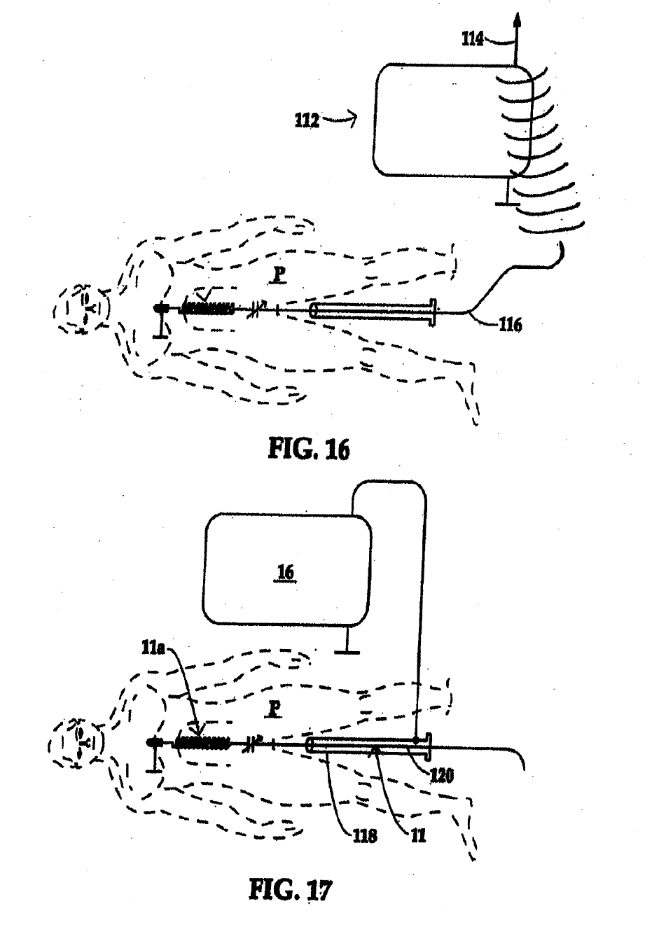

In another embodiment, a wireless coupling is accomplished with an external radio transmitter 112, as shown in FIG. 15. An antenna 114 of the external radio transmitter 112 interacts with the proximal guide wire end 116, which acts as a receiver antenna, as shown in FIG. 16. Except for the coupling through antennas this configuration functions as described for the wireless system 10 with the detector unit 16 and the guide wire 11 as shown in FIG. 1.

In yet another embodiment, the coupling between detector unit 16 and the resonance circuit 23 in the guide wire 11 is accomplished capacitively as shown in FIG. 17. An insertion sheath 118 might be equipped with a special metallic layer which acts as one capacitive electrode while the proximal guide wire section 120 inserted through the sheath acts as the opposite electrode. Instead of a special metal layer, the metallic braid many sheaths utilize for torqueabilty could be utilized.

As depicted in FIG. 18, an external or detector resonance circuit 32 may include a printed disposable coil 34 attachable to a patient near an intervention site. Coil 34 may be embedded in a strip 36 of polymeric material that is provided with an adhesive layer 38 and a removable cover sheet 40. A capacitor 42 of the LC resonance circuit 32 may be provided in strip 36 or separately therefrom.

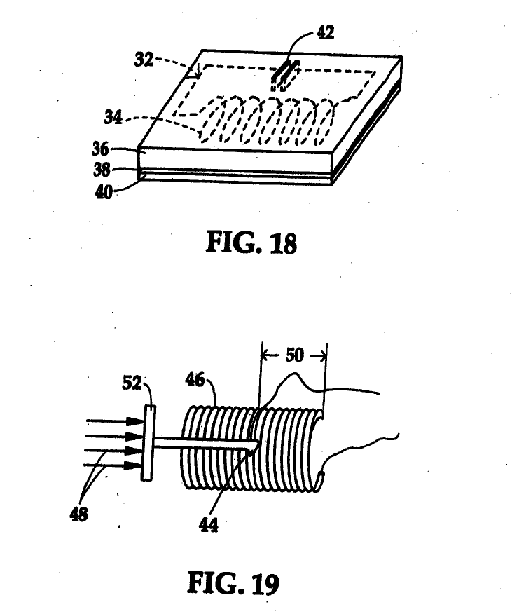

As shown in FIG. 19, a resonance circuit on a guide wire may have a movable electrical contact 44 shiftable relative to a coil 46 so that changes in pressure 48 of an external fluid (e.g., blood) results in shifting of the contact relative to the coil and changing the active length 50 of the coil, thereby varying an inductance of coil and concomitantly the resonance frequency of the resonance circuit. The movable electrical contact 44 is coupled to a plate or disk 52 that moves relative to the guide wire in response to changes in external fluid pressure 48.

1. Resonator with Capacitive Sensor

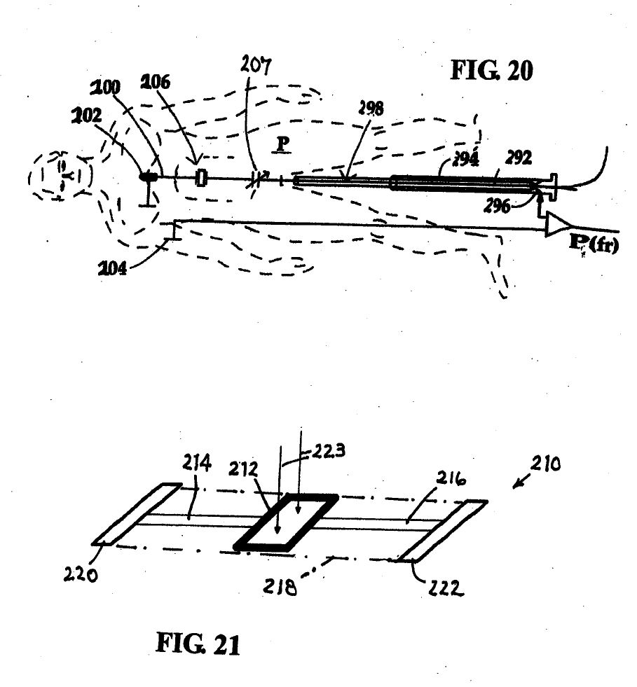

FIG. 20 shows a resonator 206 and capacitive sensor 207 connected in close proximity to each other in a distal end portion 200 of a guidewire 298, near a conductive tip 202 of the guidewire.

The resonator 206 is a ceramic element from aluminum nitride or another ceramic material which produces a resonance similar to a quartz crystal as they are used in precision oscillators. However, in contrast to a quartz crystal the resonance is usually broader and it can be pulled over a wider frequency range via a variable capacitance. Ceramic resonators can also be produced in a smaller form factor, allowing the integration into small 14/1000 inch guidewires. They are less prone to mechanical damage. Metal in close proximity will not have an adverse effect on the properties of a resonator. It is of little difference whether the sensor 207 or the resonator 206 is the more distal element. The contact to the wire can be as simple as a single pinch contact at the proximal wire end since no active supply voltage is required. The resonator 206 can alternatively be located proximally from the capacitive sensor 207.

FIG. 20 shows the guide wire 298 as having a proximal end portion 292 inside a sheath 294. A contact point 296 is in the proximal sheath portion (hub), outside the patient P. The resonance circuit includes a body contact ground electrode 204.

Another embodiment is a parallel resonant circuit where the capacitive sensor 207 is connected in parallel with the resonator 206. This is usually less advantageous than a series connection.

Yet another embodiment is a connection of the resonant circuit to the system via an additional conductor wire inside or on the guidewire (see further discussion herein below). This eliminates the need to use the patient body for ground return but may make wire production and handling more cumbersome, mainly due to the need for external contacts. The transmission method could be a central core wire coaxially inside a hypotube or it could be a differential scheme with two insulated strands in a spiral-style guidewire.

The electronic circuitry in the external system acts like a network impedance analyzer. It measures amplitude and phase of the whole guidewire assembly and determines where the resonance is found at any given time. Phase shift of the RF current into the wire versus applied RF voltage is generally a more precise method than measuring only the peak in the amplitude of the current. The location of the resonance in the frequency spectrum indicates the local pressure. Linearization is usually required.

Pressure exerted on the capacitive sensor 207 will change its capacitance. This in turn will shift the resonance of the resonator. The external system can detect such movement of the resonance by monitoring the RF current into the guidewire for phase, amplitude or both.

2. MEMS Resonator for Direct Pressure Sensing

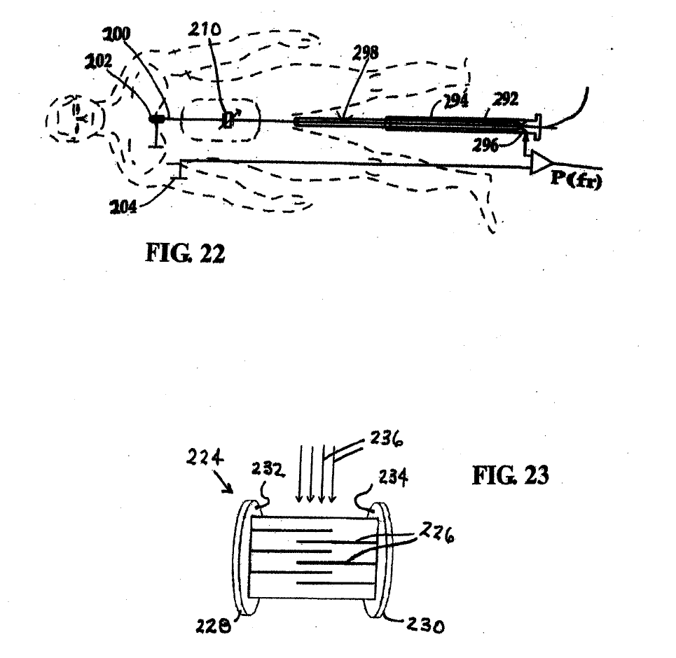

FIGS. 21 and 22 illustrate a sensor 210 which is of similar material as the sensor in FIG. 20. However, sensor 210 is longer and operates like one or several miniature tuning forks. The example shows a two-fork version similar to that proposed by Sandia National Laboratories (Olsson, December 2012) for use as an accelerometer.

The present invention intends to use the MEMS (micro-electromechanical system) sensor 210 not as an accelerometer where the mass accelerates sideways and lengthens one tuning fork while shortening the other. Instead, sensor 210 is designed to measure a pressure exerted onto a center plate 212 that holds the two ceramic tuning forks 214 and 216 together. A membrane 218 is provided to prevent contact between the ceramic and the patient's blood. Two electrodes or contacts 220 and 222 provide electrically conductive connection to the guidewire 298.

The resonance can be measured externally in the same way as discussed hereinabove. No capacitors, inductors or any other components may be necessary in the guidewire 298 when using such a resonator 210. This greatly reduces complexity and cost when producing the guidewire.

Increasing pressure 223 pushes the center plate 212 farther down which pulls on both tuning fork resonators 214, 216, stretching them. This causes their resonant frequency to shift and such shifts can be detected by the external system. The resonator 210 needs to be of reasonably low impedance so that large parasitic capacitances from the insulated part of the guidewire 298 to the surrounding blood will not weaken the detection of the resonance excessively.

Another embodiment is a connection of the MEMS sensor to the system via an additional conductor wire inside or on the guidewire (discussed in detail hereinafter). This eliminates the need to use the patient body for ground return but can make wire production and handling more cumbersome. The transmission method could be a coaxial central wire inside a hypotube or it could be a differential scheme with two insulated strands in a spiral-style guidewire.

3. Ceramic Pressure Sensing

FIG. 23 shows a sandwiched ceramic structure 224 having multiple capacitive plates 226 (exemplarily of nickel) in an interleaved array between two conductive epoxy panels 228 and 230 having copper terminations 232, 234, forming a multilayer ceramic capacitor or MLCC. Such a multilayer ceramic capacitor 224 is produced by AVX/Kyocera. A typical ceramic material is barium titanate. Many such capacitors have the undesired side effect of being microphonic. When exposed to an AC voltage they can emit audible noise. Since the effect is reciprocal an external pressure wave 236 can alter the capacitance and also generate an AC voltage. This capacitance change or the voltage can be sensed by electronics in many ways, for instance, either directly as a generated AC signal or indirectly by using the capacitor 224 inside a resonant circuit, where the inductive component can be disposed far away from the capacitor (for example outside of the guidewire inside the detector system) if the capacitance is large enough.

Due to the proliferation of miniaturized electronics such as cell phones these ceramic structures are being made available in ever smaller and higher capacitance variants, the goal of the industry being to provide a higher density of capacitance per volume. The number of layers is, therefore, increasing. Aiding this trend is the fact that supply voltages of modern ICs are dropping to lower values, thus requiring less breakdown voltage rating of capacitors. That is advantageous for this invention as it reduces the source impedance of the pressure-induced capacitive change signal and thus increases the chance of only needing this one capacitive element in the guidewire as a sensor. The signal could be extracted using the same methods as described above with reference to FIGS. 1-19. The electronic system could measure capacitance, generated signal, or both.

Where multilayer ceramic capacitor (MLCC) 224 is used in a guidewire-carried pressure-measuring LC circuit as disclosed above with reference to FIGS. 10-19, the MLCC 224 is preferably a very high density MLCC with consequently high capacitance. Such a high capacitance requires a large inductance which would be difficult if not impossible to integrate into a small guidewire. A solution is to split off and place the bulk of the inductance outside the guidewire. This same design can be used in any guidewire-mounted circuit having an inductance that need be partially located at the distal end of the guidewire.

4. FFR Electronic System

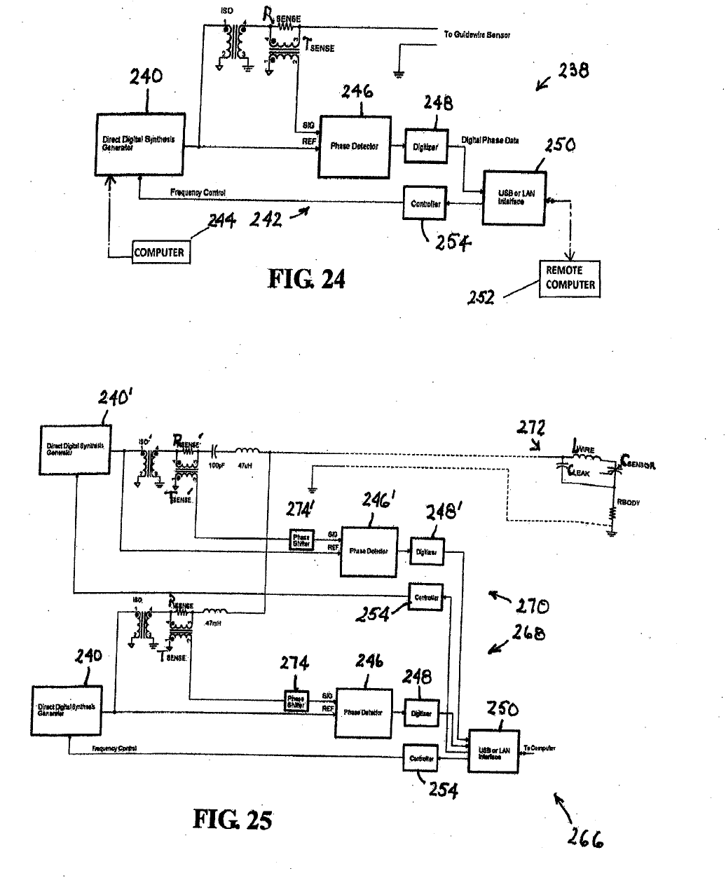

FIG. 24 depicts one embodiment of an electronic system 238 suitable to detect resonances in a resonator 206, 210 and also in an inductor-capacitor based FFR guidewire (FIGS. 1-19). Description below refers specifically to the guidewire systems of FIGS. 20 and 22. Only important components are shown, support functions such as power supply or computer algorithms being known to engineers skilled in the art.

A controllable RF generator 240 sends an RF signal of fixed frequency into the guidewire 298 and a sensing circuit 242 measures the phase shift between the oscillator output and the current the wire draws. The generator 240 can be of any kind that can be controlled via an analog or digital system. Phase-locked loop (PLL) used to be common but due to faster control of the frequency direct digital synthesis (DDS) has become a more contemporary method. The current is sensed at RSENSE and sent via transformer TSENSE for safety isolation purposes.

A computer 244 commands the controllable generator 240 to move to a certain frequency that is guaranteed to be lower than the resonance in the guidewire 298. Computer 244 then commands generator 240 to increase its frequency incrementally until a desired phase shift between the generator output and the current sense signal tapped off at RSENSE has been reached. This phase shift can later be adjusted again to compensate for capacitive drift in the various leakage capacitances from the guidewire to its surroundings.

A phase detector 246 measures the phase shift and its analog output is digitized by a converter or digitizer 248. The phase value moves with pressure but not in a linear relationship. A two-way universal serial bus (USB) or local area network (LAN) interface 250 communicates with a computer 252 (optionally the same as computer 244). Computer 252 may perform the functions of computer 244 via the interface 250 and a controller 254. The computer 252 linearizes the phase signal over pressure and displays the pressure in a rolling graph or in any other desired form. A LAN interface 250 may be advantageous because it electrically isolates as well as allows the computer 252 to be at a remote location, for example in a shared control room that often exists between two catheter labs in a hospital. Existing hospital infrastructure can then be used for data transfer. The bandwidth of the data is very low compared to other usual activities, less than 5 kbit/sec.

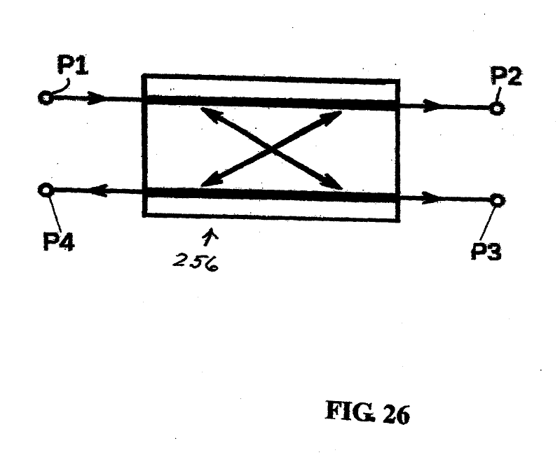

Another embodiment of the electronics is to only measure amplitude, by using a directional coupler 256 (FIG. 26). This can be sufficient when using resonators with very narrow frequency response. The directional coupler 256 is connected at a receive or input port P1 to a fixed-frequency generator, for instance, of the direct digital synthesis type. At a transmit or output port P2, the directional coupler 256 is connected to a guidewire containing a resonator (as shown in FIGS. 20 and 22). At an isolated port P4, the directional coupler 256 is connected to a signal-processing computer for monitoring amplitude changes. Another port P3 of the directional coupler 256 is not used in this application. Ground return is common to all ports.

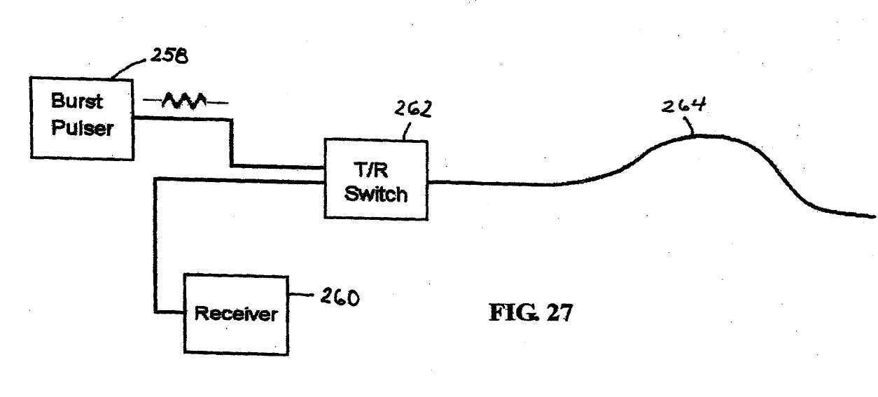

Yet another embodiment (FIG. 27) of the electronics is to operate in the time domain. Here, a pulse oscillator 258 is used that only sends out bursts. A receiver 260 of the system then listens for the ringing from the resonant circuit in a guidewire (264). This method can prove beneficial if there are interference concerns with the above continuous wave method. If the resonance is at a suitable frequency the burst oscillator 258 could operate in a license-free industrial-scientific-medical (ISM) band. FIG. 8 shows burst pulse generator 258 and receiver/processor circuit 260 alternately connectable to the guidewire 264 via a transmit/receiver switch 262.

A further electronic processing system 266 shown in FIG. 25 compensates for changes in leakage capacitances that occur naturally between a coated guidewire and the body of a patient or subject. These capacitances change when the wire is moved or pushed back and forth.

A first section 268 of processing system 266 is, as in the embodiment of FIG. 24, the phase detection circuit that senses pressure. Phase detection circuit 268 has components identical to those in FIG. 24 and bearing the same reference designations. Processing circuit section 268 is operated at a frequency much lower than that of a second circuit section 270 and is designed to be insensitive to frequencies used in the second section 270. Wire movements will cause changes in the leakage capacitance C_{LEAK} in a guidewire 272, resulting in false pressure change indication. Filters to set the spectral sensitivities of the two sections have been omitted from the drawing for clarity and are easy to design by anyone skilled in the art, since essentially they are just LC filters. Phase shifters 274 and 274′ are needed because most ordinary phase detection circuits suffer from ambiguity every 180 degrees and also from inaccuracy when operated too close to 180 degrees in phase shift and multiples thereof.

The second or upper circuit section 270 in FIG. 25 has components that are analogous to respective components of the circuit of FIG. 24 and bear the same reference designations with a prime mark. Circuit section 270 runs at a much higher frequency, typically above 1.6 MHz to avoid noise from the AM radio band. Because of the inductor L_{WIRE} in the guidewire 272 the second circuit section 270 will largely be sensitive only to the leakage capacitance C_{LEAK} but not the pressure-measuring capacitance C_{SENSOR}. Therefore, the phase information gathered in the upper or section circuit section 270 will indicate the leakage capacitance C_{LEAK}. This information can then be used in the system software to compensate for the amount of false pressure change information caused by a change in the leakage capacitance C_{LEAK}. This essentially neutralizes changes in pressure measurements due to changes in leakage capacitance C_{LEAK} and greatly improves the pressure reading accuracy of the system in a clinical setting, where wire movements are part of the routine procedure of measuring a fractional flow reserve.

Because the computational overhead and the data rates are low it is also possible to use a hand-held device such as a smart phone or tablet computer. Even transmission of the data through regular digital voice data channels (cell networks) is feasible. This can open up options if the technology is considered for other purposes such a battlefield use.

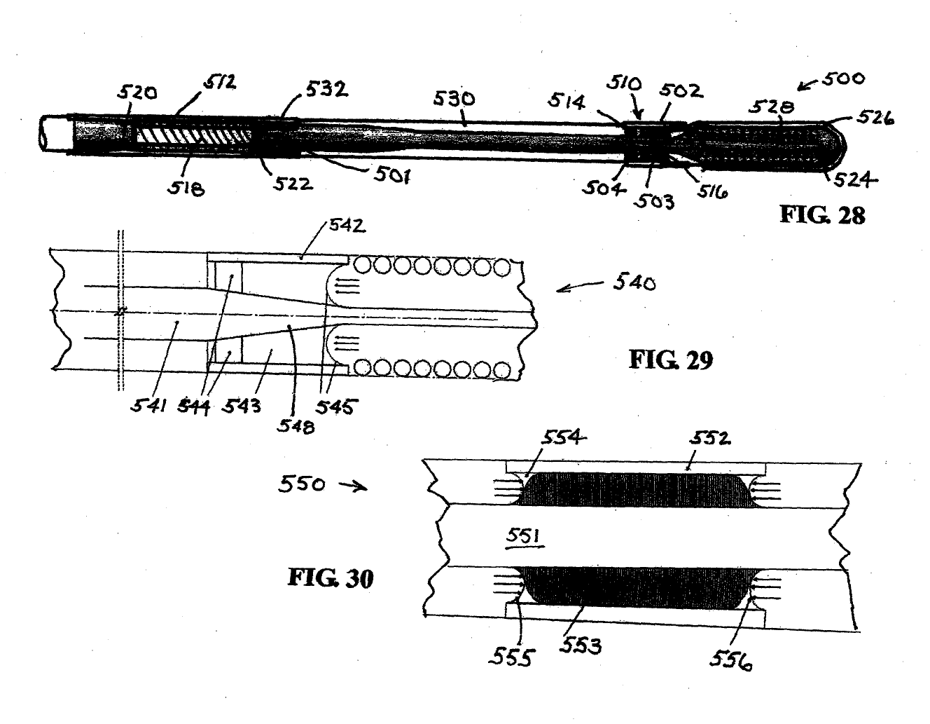

As depicted in FIG. 28, a capacitive pressure sensing guide wire 500 comprises a guide wire core wire 501 having a tubular capacitive sensor 510 and a coil 512 at its distal end portion. The cylindrical shaped sensor 510 utilizes core wire 501 as the inner sensor electrode. A tubular polymer member 502, metalized on the inside, acts as the outer electrode and pressure sensing membrane. Preferably this tubular polymer member 502 has a variable wall thickness to enable the cylinder to take an oval or ovoid shape when pressure is applied. This way the sensitivity of the sensor 510 to pressure changes will be increased. Between inner electrode or core wire 501 and tubular outer electrode 502, an electrolyte 503 is disposed. An air gap 504 allows the areas of contact between the electrolyte 503 and the outer electrode 502 and inner electrode 501 to vary depending on applied pressure at the outside electrode 502. Electrolyte 503 and air gap 504 are enclosed or bounded by a pair of polymeric spacer rings 514 and 516. The guide wire 500 may be inserted in the body of a patient or subject through blood vessels and vascular structures, such as to the site of a damaged or diseased blood vessel, as typically performed in interventional cardiology, without having to disconnect a contact handle from the proximal wire portion first. Catheters may be guided over the guide wire in the patient's body.

As further depicted in FIG. 28, coil or inductor 512 is provided with a ferrite core 518 and is disposed between separated sections of core wire 501. Coil 512 is electrically linked to the sections of core wire 501 by connectors 520 and 522. A distal tip 524 of guidewire 500 have a polymer coating 526 which is metalized for conduction all around the distal tip if warranted. Otherwise the distal tip 524 has the same construction as conventional interventional guide wires, including a floppy coil structure 528.

Tubular polymeric member 502, with its metalized inner diameter, may be cut at an angle to allow ease of electrical bonding like a pad. Guidewire 501 is provided with a polymer coating 530 at least between coil 512 and capacitive sensor 510 so that the coil on the distal side only acts as ground electrode. An outer connection 532 may be a Kapton tube disposed over coil 512 and bonded to core wire 501.

FIG. 29 illustrates a differently configured capacitive sensor 540 for the distal portion of the guide wire 500. Here the capacitive sensor 540 comprises a core wire 541 having a conical portion 548 and forming an inner electrode of the sensor. An outer electrode 542 is a tubular member essentially fixed in shape so as to not deform under surrounding blood pressure. A pressure sensitive membrane 545 is mounted in a transverse or cross-sectional fashion at a distal or front end of the sensor 540. Pressure 546 applied to this membrane 545 will deform the membrane and thereby modify the volume occupied by electrolyte 543. Capacitance changes because of variation in the area of electrolyte/electrode contact, variously compressing an air volume 544. The capacitive change is enhanced through the conical portion 548 of the inner electrode 541 which will cause more surface variation (electrolyte/electrode) per volume movement.

The position of the coil 512 and sensor 510 or 540 in the distal portion of the guide wire 500 may either be as shown in FIG. 28, in which the coil 512 is positioned more proximal than the sensor, or vice versa.

FIG. 30 shows yet a different capacitor configuration 550 with an isometric core wire 551. Pressure sensing membranes 555, 556 are mounted proximal and distal of the cylindrical capacitor 550. The membranes 555, 556 are variably deformed in accordance with the magnitude of ambient blood pressure, thereby varying the electrolyte volume 553 (vs. the volume of one or more air pockets 554) to change the area of contact between the electrolyte 553 and electrode 552. An outer tubular electrode 552, metalized along an inner surface, does not change its configuration in response to changes in ambient pressures.

In yet another embodiment the space between an outer electrode and an inner electrode formed by a guide wire core wire is minimized to 100 microns diameter or less and the electrolyte is mainly stored in a pressure sensitive volume section proximal or distal (or alternatively both) to the capacitor. The pressure sensitive volume is connected with the capacitor so that the electrolyte can move into the space between the outer electrode and inner electrode of the capacitor when the pressure sensitive reservoir(s) is compressed. This construction will allow an even further increased sensitivity compared to the structure of FIG. 29.

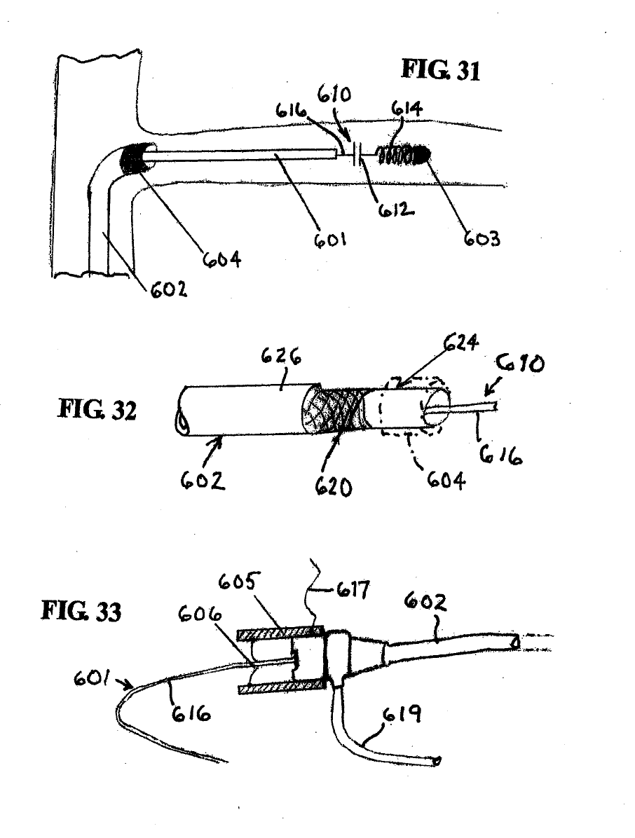

With reference to FIG. 31, the present invention comprises a guide wire 601 having a resonance circuit 610 consisting of a capacitive sensor 612 and a coil 614 at its distal end portion. The resonance circuit 610 is connected to a conductive tip or ground electrode 603 which electrically connects the resonance circuit with the bloodstream of the patient. Through the bloodstream connection is made to another ground electrode 604 mounted on the distal portion of a sheath or guiding catheter 602. In this embodiment of the invention, the resistance between the ground electrodes 603 and 604 is minimized since the blood path consists of a relatively short length of approximately 5 to 20 cm depending on lesion location and since blood is a better conductor than tissue. Besides the minimized electrical resistance compared to an approach with an external ground electrode this approach offers the convenience of not having to attach an external ground electrode to the patient making the procedure easier and faster. Last but not least this approach offers the advantage of a short connection through the patient's bloodstream without having vital organs like the heart being part of the conductive path way. This is problematic especially in an approach where the sensor is actively powered as described in US Patent Application Publication No. 2001/0051769/A1 and U.S. Pat. No. 7,645,233 B2. In a different embodiment the ground electrode (604) at the distal sheath or guide catheter end can be mounted on the distal end of a flexible tube which is inserted into the sheath or guide catheter (602). This obviously has the advantage that any type of sheath or guide catheter, the user prefers, can be utilized.

FIG. 35 shows the electrical connections of the overall system consisting of circuit path Z-Blood between the ground electrodes 603 and 604, the resonant circuit 610 connected to a core wire 616 of the guide wire 601 on one side and the distal guide wire ground electrode 604 (either mounted distally on the sheath or guide catheter or a tube to be inserted into either the guide ort sheath) on the other side. A phase detection system 618 is connected to the distal ground electrode 604 of the guide catheter or sheath 602 and the core wire 616 of guide wire 601 and is described in detail hereinabove with reference to FIGS. 20-27.

FIG. 33 shows one way of connecting the core wire 616 of guide wire 601 with the FFR (Fractional Flow Reserve) system through a brush contact 606 at the proximal end or hub of the sheath or guide catheter 602. The brush contact 606 is part of an attachment or coupling 605 which is designed to plug onto the hub of the sheath or guide catheter 602. A lead or wire 617 extends from the attachment or coupling 605 to the FFR system. A liquid conduit or tube 619 extends to the hub 622 for conducting a fluid flush for the proximate pressure sensor.

Alternatively, the brush contact 606 can be integrated into the hub. In yet another embodiment the brush contact could be mounted proximally into a tube to be inserted into the guide or sheath 602.

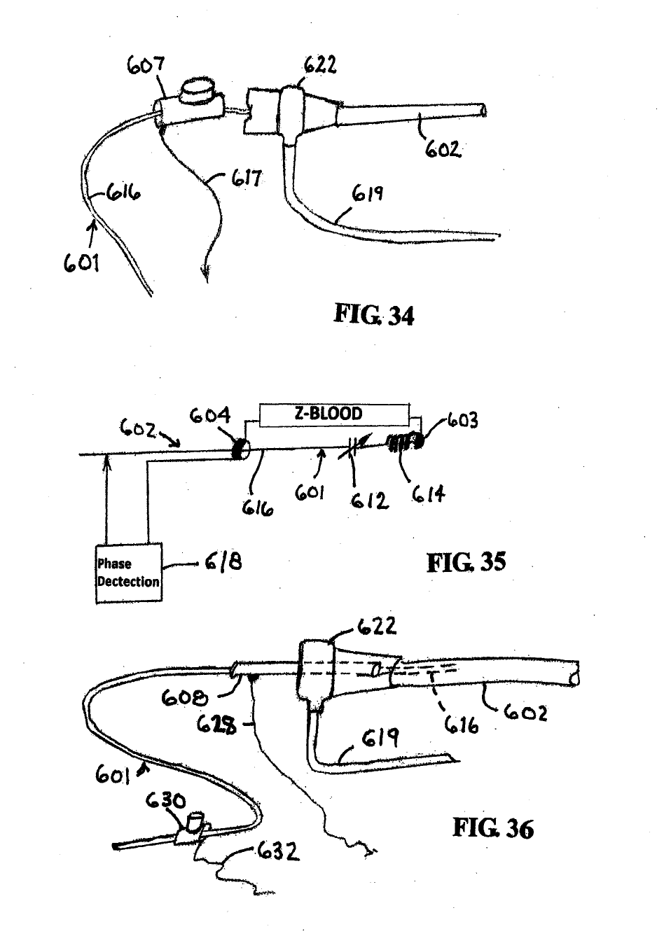

FIG. 34 shows a different embodiment where the electrical connection to the FFR system is made through a wire torquer 607 attached to the proximal end of the core wire 616 of the guide wire 601.

FIG. 32 shows another embodiment of electrically coupling the core wire 616 of guide wire 601 with the sheath or guide catheter 602. In this case coupling is achieved capacitively, between a stainless steel braid 620 as one capacitor electrode and core wire 616 of guide wire 601 as the opposite capacitor electrode. Braid 620 is sandwiched between an inner layer 624 of polytetrafluoroethylene or other polymeric material and an outer layer 626 of soft nylon or similar polymeric material. Two mutually insulated conductors (not shown) extend along the sheath or guide catheter 602 to the FFR system from the blood electrode 604 and the stainless steel braid 620, respectively. Instead of utilizing the braid 620 of the sheath or guide 602 a metalized tube (not shown) could be inserted to create the opposite electrode to the core wire 616 of the guide wire 601.

The guide wire 601 may be inserted in the body of a patient, for instance, through blood vessels and vascular structures to the site of a damaged or diseased blood vessel, as typically performed in interventional cardiology. Catheters may be advanced over the guide wire so inserted. The capacitive coupling approach from FIG. 32 and the sheath brush contact 606 shown in FIG. 33 allow insertion of catheters without having to disconnect an electrical contact handle from the proximal guide wire end. This allows for the FFR measurement to fit seamlessly into the interventional procedure.

The position of the coil 614 and capacitive sensor 612 in the distal portion of the guide wire 601 may either be as shown in FIG. 31, in which the coil 614 is more distal than the sensor 612, or vice versa. The coil 614 provides an inductance which may utilize the coil tip (or sections thereof) at the distal end of the guide wire 601, often referred to as the floppy tip (528, FIG. 28). This inductor 614 and pressure sensitive capacitor 612 create the resonance circuit 610 with a resonance frequency varying with blood pressure fluctuations. As described above with reference to FIGS. 28-30, a typical vacuum or air filled capacitor cannot drive the load represented by body tissue. In order to fit the minimal dimensional requirements of a typical 14/1000 guide wire and to provide enough capacitive change detectable through body and core wire conduction, an electrolyte capacitor 510, 540, 550 is utilized. In another embodiment the capacitor 612 can be of fixed value while the inductance of the coil 614 changes according to the surrounding blood pressure as described hereinabove with reference to FIGS. 1-19. In yet another embodiment the resonance circuit 610 can be replaced by the ceramic resonator 206 which varies in resonance frequency depending on the surrounding blood pressure as described hereinabove with reference to FIGS. 20-27.

The blood pressure monitoring process may be done periodically during interventional procedures or as needed to classify the hemodynamic significance of a lesion, so that the blood pressure about the site of intervention can be accurately measured.

From the foregoing description of FIGS. 31-36, it will be apparent that there have been provided a quasi wireless pressure sensing guide wire and detector. Variations and modifications in the herein described apparatus, method, and system in accordance with the invention will undoubtedly suggest themselves to those skilled in the art.

FIG. 31 demonstrates the utilization of typical guide wire components as electrical conductors to avoid having to integrate additional electrical wires or other signal communication means into the guide wire structure which negatively affects wire handling. The compromised wire handling of the commercially available pressure sensing guide wires represents a significant barrier towards widespread use of pressure sensing guide wires. As FIG. 31 demonstrates, the wire handling can be equal to non-pressure-sensing guide wires by requiring only 2 electrical conductors, utilizing the standard wire components, core wire and distal tip.

FIG. 35 shows the electrical configuration which to the user appears wireless since the proximal guide wire end does not need to be connected with a connector handle. Instead the sheath or guide catheter 602 which is part of any interventional procedure contains a brush contact 606 as shown in FIG. 33, to connect to the proximal end of the wire 616, while the distal end of the wire is in electrical contact with the patient who is connected to ground potential through a ground electrode 604 mounted to the distal end of the sheath or guide catheter 602 as shown in FIG. 31. External patch electrode grounding techniques are widely utilized in RF ablation procedures with a typical impedance of about 100 Ohms from RF electrode to ground. The other end of the resonance circuit 610 is connected to the wire body or core wire 616. The proximal end portion of the wire 616 is not insulated in order to make contact with the contact brush 606 within the sheath 602 as shown in FIG. 33. This has the advantage that wire handling is not compromised since standard wire components (core wire and distal tip) are utilized as electrical conductors avoiding the insertion of additional electrical wires.

FIG. 36 shows yet another configuration where the ground connection is established through a conductive cylinder or tube 608 inserted into a sheath or guide hub 622 to make contact with the fluid column inside the sheath or guide 602 and therewith the patient's bloodstream. Compared to the approach shown in FIG. 31, this has the advantage that the sheath 602 does not need to be modified with a permanent ground electrode (604). Cylinder or tube 608 can be either a conductive cylindrical tube advanced over the proximal wire end into the hub of sheath 622 or guide catheter 602 or consists of 2 linked half shells so that the cylinder can be opened and closed around the guide wire. In either case the cylinder or tube 608 is connected through a wire 628 to the ground terminal of the FFR system. FIG. 36 also depicts a coupling through a wire torque 630 electrically linked on the one side to guide wire core wire 616 and on the other side to the FFR system via a wire 632.

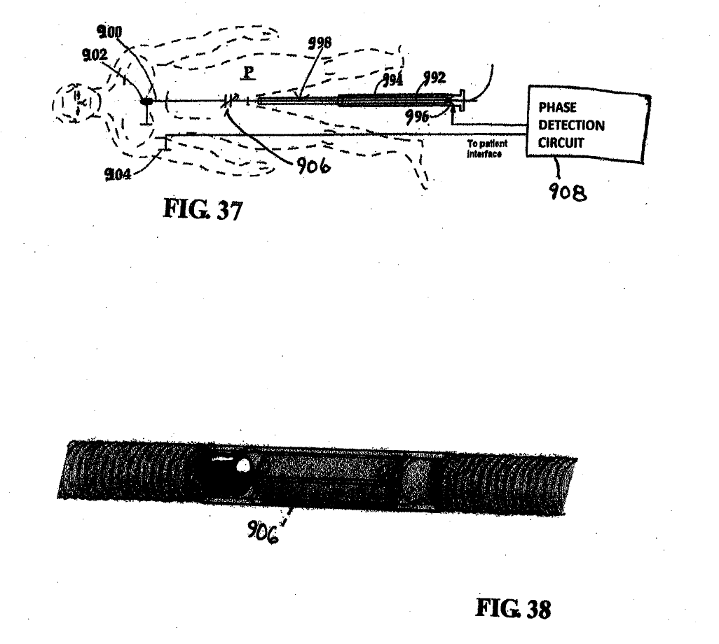

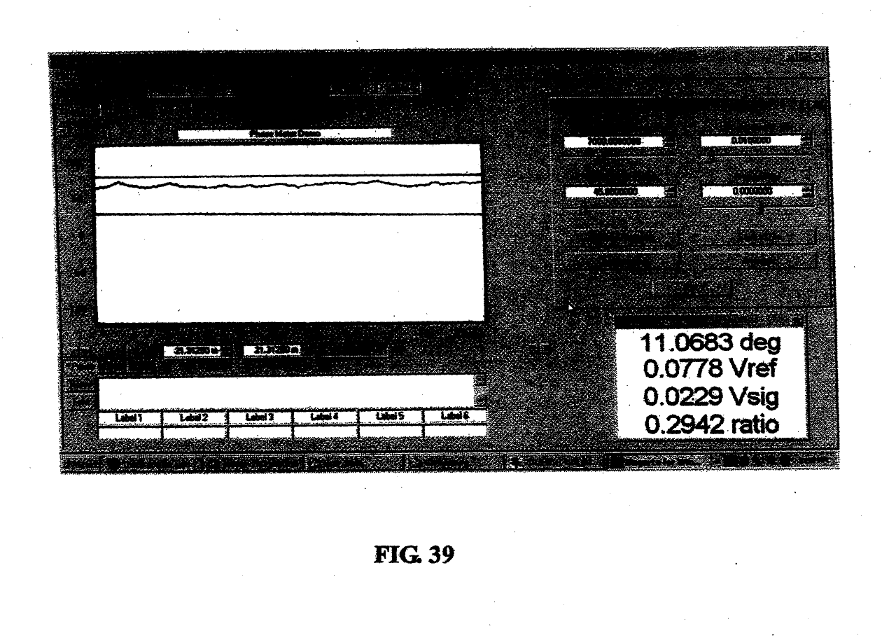

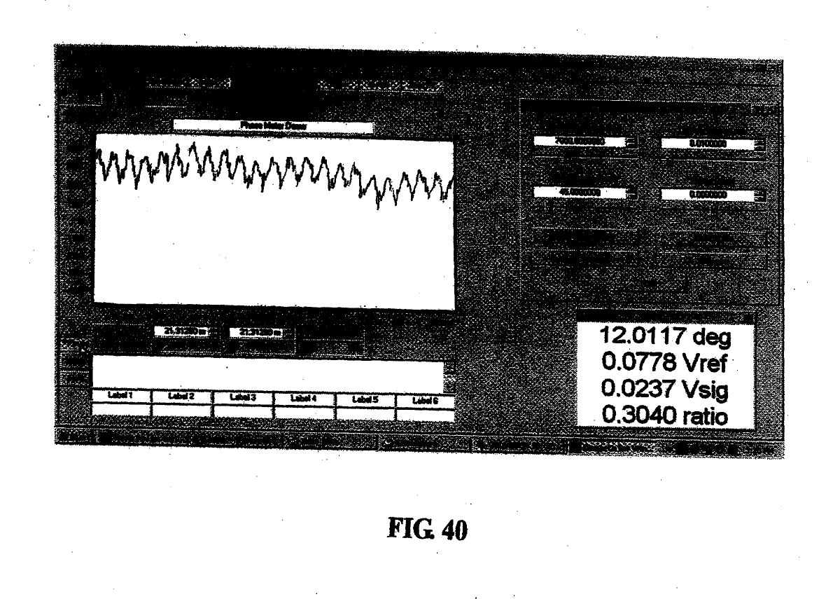

As depicted in FIG. 37, the present invention comprises a guide wire 900 having a capacitive sensor 906 (FIG. 38) at its distal end portion. The capacitive sensor 906 is connected to a conductive tip or ground electrode 902 which electrically connects the capacitive sensor with the bloodstream of the patient. Through the bloodstream connection, is made to an external ground electrode 904 attached to the patient. Proximal to the sensor the guide wire is electrically isolated from the body with a thin layer or coating of insulating material. In this embodiment of the present invention the impedance between the ground electrodes 902 and 904 is on the order of less than 30 Ohms. Values up to several hundred Ohms can be tolerated for this invention so that even smaller patch electrodes than used in ablation procedures are feasible. This serial impedance might be problematic in an approach where the sensor 906 is actively powered as described in US Patent Application Publication No. 2001/0051769 A1 and U.S. Pat. No. 7,645,233 B2 but is not of concern in this invention. Another critical parasitic factor is the wire/body (blood) impedance. The capacitive sensor 906 needs to have about an order of magnitude higher capacitance than the parasitic wire/body capacitance. As can be seen from FIG. 39 this was verified in vivo to be the case with a wire/body capacitance in the 300 to 400 pF range. Also variations of this parasitic capacitance with wire movement on the order of 30 pF (see middle trace in FIG. 39) are of no concern as long as the sensor is producing a capacitive change in the nF range. FIG. 40 shows the impact of heartbeat and breathing on the parasitic capacitance. This variation is in the pF range and again, will not impact the pressure measurement given a capacitive change in the nF range. FIG. 37 shows the electrical connections of the overall system consisting of the blood-body circuit path Z-Blood/Body between the ground electrodes 902 and 904, the capacitive sensor 906 connected to the core wire of the guide wire 900 on one side and the distal guide wire ground electrode 902 on the other side. A phase detection circuit component 908 is connected to the external patient ground electrode 904 and the guide catheter or sheath 994 through a contact 996 or directly with the proximal core wire 996 of the guide wire 900.

Phase detection circuit 908 essentially operates like a network impedance analyzer to detect the capacitive changes. It measures phase and amplitude in very fast sequence, typically 100 times per second or more. In contrast to a classical impedance analyzer phase detection circuit 908 measures the whole frequency spectrum of interests not in sweeps but simultaneously, typically 2 kHz to 10 kHz. Phase detection circuit 908 uses the complex Fast Fourier Transform method (FFT) or similar calculations.

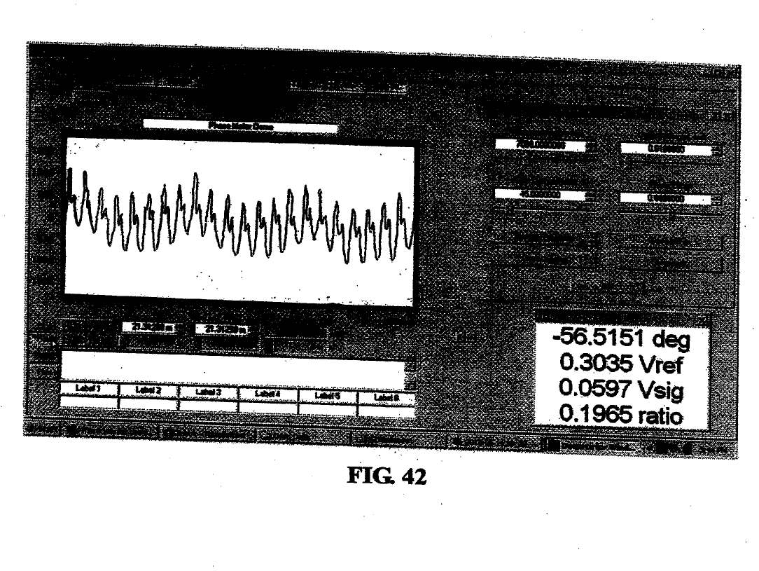

FIG. 42 shows the impedance change with cardiac and breathing cycles and is an amplitude instead of a phase measurement. This is useful for two purposes. The system must automatically find a frequency where a change in impedance has the least impact on the displayed pressure value and monitoring this amplitude signal enables it to do so as needed. In addition, this signal allows a basic monitoring of vital signs in the absence of other suitable gear, for example in emergency care.

The guide wire 900 may be inserted in the body of a patient. Catheters may be guided over the guide wire 900 inserted in the patient's body through blood vessels and vascular structures, such as to the site of a damaged or diseased blood vessel, as typically performed in interventional cardiology. The sheath brush contact 996 shown in FIG. 37 allows insertion of catheters without having to disconnect an electrical contact handle from the proximal guide wire end. This allows for the FFR measurement to fit seamlessly into the interventional procedure. Alternatively a clip contact is easily removed and attached to the proximal wire end. Guide wire torquing handles are also routinely used and these can be employed to simultaneously provide the proximal electrical wire contact.

The position of the capacitive sensor 906 in the distal portion of the guide wire 900 may either be as shown in FIG. 38 in which the sensor is positioned in the coil section of the guide wire, often referred to as the floppy tip. A typical vacuum or air filled capacitor with capacitive change in the pF range cannot drive the load represented by body tissue. In order to fit the minimal dimensional requirements of a typical 14/1000 guide wire and to provide enough capacitive change detectable through body and core wire conduction, an electrolyte-containing capacitor is utilized such as capacitor 510, 540, or 550, described hereinabove with reference to FIGS. 28-30.

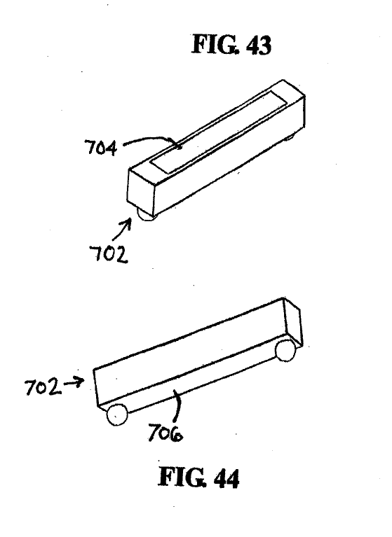

As depicted in FIGS. 43-46, a MEMS sensor 702 for implementing capacitive sensor 906, or alternatively any of the variable-capacitance capacitive sensors disclosed herein, comprises two capacitors 704 and 706 in parallel, with exemplary dimensions of 0.2×0.2×1.2 mm (width, depth, length) and a capacitance range of 0.5-5 nF. Improved dielectrics produced over time may result in an even higher capacitance range. Two plates 708 and 710 (FIG. 47) of the capacitor 704 (FIG. 46) in the form of ion implanted electrodes are separated by a specified air or vacuum gap 712 and one plate of the capacitor 704 is held fixed (bottom plate 710) while the other plate deflects with applied pressure (top plate 708). In between the two plates are disposed air gap 712 (air, vacuum or ideal gas) and a dielectric 714 with a high dielectric constant (2000-15000 or more). The dielectric 714 can be BaTiO3, CaCu3Ti4O12 or a similar material. As pressure is applied the top plate 708 deflects through the air gap 712 until it makes contact with the dielectric layer 714. Once the top plate 708 makes contact with the dielectric 714 the capacitor 704 turns on. As pressure is increased, the area of contact of the top plate 708 with the dielectric 714 increases. The purpose of the dielectric 714 is to significantly increase the capacitance achievable between the top and bottom electrodes 708 and 750. The capacitance prior to top plate and dielectric contact is negligible. The relationship for capacitance with a dielectric is provided by the following equation:

Capacitance=(er*e*A)Ih

where e is permittivity, er is dielectric constant, A is plate area and h is distance between plates. The minimum pressure range of the device is a specified by a minimum area of contact between the top plate 708 and the dielectric layer 714. The maximum capacitance is defined when a saturation pressure is reached and maximum area of contact is achieved. As the area of contact changes between the top plate 708 and the dielectric layer 714, the capacitance changes and this change in capacitance is proportional to applied pressure. This physical phenomenon is identical for the second capacitor 706.

The electrodes of the top and bottom plates 708 and 710 can be fabricated by doped silicon, platinum or another suitable material. The top plate 708 is fabricated using a reactive ion etch (RIE) process to etch a diaphragm (0.7-3 micron thick) into the membrane of an SOI (silicon on insulator) wafer. The resulting standoffs create the separation between the two plates and define the gap 712 between the top plate electrode 708 and dielectric layer 714. An alternative method to creating the standoffs is depositing or growing an oxide layer and patterning it via photo lithography and etching means. The handle portion of the SOI wafer is present to improve handling robustness during sensor fabrication and is greater than 100 microns in thickness but in a preferred embodiment is about 300 microns thick. The bottom plate 710 and the dielectric layer 714 are located on a bulk or SOI silicon wafer 716. The bottom plate electrode 710 is fabricated by doping the silicon wafer or depositing a platinum or other suitable metal on the wafer. The dielectric layer 714 is deposited over the bottom plate electrode 710. The first wafer containing the top plate 708, electrode and standoffs is then bonded to the second wafer containing the bottom plate 710, electrode and dielectric layer 714. A similar procedure is followed to fabricate the second capacitor 706 with the added steps of fabricating the through silicon vias for electrical interconnection.

In a preferred embodiment, a fusion bond is used to bond the two wafers creating a single capacitive sensor 702. In order to create a good fusion bond, a thin oxide layer is grown on both the first (top) and secondary (bottom) wafers. This oxide layer is preferably 500 angstroms or less. However alternate means such as glass frit or elastomeric materials can be used to bond the two wafers together.

In the preferred embodiment the top plate electrode is fabricated by doping the silicon membrane via ion implantation or diffusion after the first and second capacitors 704 and 706 are fusion bonded together and the handle wafers and oxide have been removed via dry or wet etch. This can be accomplished because the membrane is, for example, 1-2 microns thick. This is repeated for the second capacitor. An alternative to doping silicon for the top electrodes is depositing platinum or other suitable conductive material prior to fusion bonding.

In the preferred embodiment, electrical interconnects are on one side (top or bottom) of the sensor. A suitable metallization and barrier is deposited or plated for wire bonding, solder bumps, silver epoxy other electrical interconnect means. An alternative to using electrical interconnects is a wireless communication system such as but not limited to an inductive coil and needed electrical circuitry for resonance frequency shift telemetry as described above.

On the opposite side of the electrical interconnects, the oxide from the SOI wafer is left intact on the edges of the sensor with the center portion over the capacitor diaphragm removed. This creates a short standoff that will prevent mounting tools like a vacuum tip from touching the sensitive diaphragm when the sensor is attached to the guide wire or other medical tools. The capacitors 704 and 706 are connected in parallel and their electrical signal is channeled to one or two sides of the sensor 702 by through silicon vias 718.

In yet another embodiment the capacitive sensor 906 can take the form of a ceramic resonator, that is, a multilayer ceramic capacitor 224 or MLCC as discussed above with reference to FIG. 23. Such a multilayer ceramic capacitor is produced by AVX/Kyocera. A typical ceramic material is barium titanate. Many such capacitors have the undesired side effect of being microphonic. When exposed to an AC voltage they can emit audible noise. Since the effect is reciprocal an external pressure wave can alter the capacitance and also generate an AC voltage. This capacitance change or the voltage can be sensed by electronics in many ways. Either directly as a generated AC signal or indirectly by using this capacitor inside a resonant circuit, where the inductive component could remain far away from the capacitor (for example outside of the guidewire inside the detector system) if the capacitance is large enough.

Due to the proliferation of miniaturized electronics such as cell phones these ceramic structures are becoming available in ever smaller and higher capacitance variants. The goal of the industry is to provide a higher density of capacitance per volume. The number of layers is, therefore, increasing. Aiding this trend is the fact that supply voltages of modern ICs are dropping to lower values, thus requiring less breakdown voltage rating of capacitors. That is advantageous for this invention as it reduces the source impedance of the pressure-induced capacitive change signal and thus increases the chance of only needing this one capacitive element in the guidewire as a sensor.

The blood pressure monitoring process may be done periodically during interventional procedures or as needed to classify the hemodynamic significance of a lesion, so that the blood pressure about the site of intervention can be accurately measured.

From the foregoing description, it will be apparent that there have been provided a quasi wireless pressure sensing guide wire and detector. Variations and modifications in the herein described apparatus, method, and system in accordance with the invention will undoubtedly suggest themselves to those skilled in the art.

FIG. 37 demonstrates the utilization of typical guide wire components as electrical conductors to avoid having to integrate additional electrical wires into the guide wire structure which negatively affects wire handling. The compromised wire handling of the commercially available pressure sensing guide wires represents a significant barrier towards widespread use of pressure sensing guide wires. As FIG. 37 demonstrates the wire handling can be equal to non pressure sensing guide wires by requiring only 2 electrical connections, utilizing the standard wire components, core wire and distal tip.

Patch electrode (904) grounding techniques are widely utilized in RF ablation procedures with a typical impedance of about 100 Ohms from RF electrode to ground. The other end of the resonance circuit is connected to the wire body or core wire. The proximal end portion of the wire is not insulated in order to make contact with the contact brush within the sheath as shown in FIG. 39. This has the advantage that wire handling is not compromised since standard wire components (core wire and distal tip) are utilized as electrical conductors avoiding the insertion of additional electrical wires. Alternatively a clip contact or wire torquer contact can be employed. Another contacting method could be a conductive sterile liquid or gel type contact sleeve because the allowed contact resistance can easily be 100 Ohms or more.

Phase detection circuit 908 may typically comprise an electronic signal processing circuit configured for monitoring electrical-current phase changes. The signal processing circuit preferably includes an oscillator, a current sensor, a phase detector, a digitizer and an interface, the interface being operatively connectable to a computer device. The oscillator may be a direct digital synthesis generator.

Phase detection circuit 908 measures phase of the whole guide wire assembly and determines the capacitance of capacitive sensor 906 at any given time, which indicates the local pressure. Linearization may be required. The oscillator is an RF generator which sends an RF signal of fixed frequency into the guide wire 900 and circuit 908 measures the phase shift between the oscillator output and the current the wire draws. The oscillator/generator can be of any kind that can be controlled via an analog or digital system. Phase-locked loop (PLL) used to be common but due to faster control of the frequency direct digital synthesis (DDS) has become a more contemporary method.

Phase detection circuit 908 may include a computer or microprocessor that commands the controllable oscillator/generator to move to a certain frequency. The computer or microprocessor then commands the oscillator/generator to increase its frequency incrementally until a desired phase shift between the generator output and the current sense signal has been reached. This phase shift can later be adjusted again to compensate for capacitive drift in the various leakage capacitances from the guide wire 900 to its surroundings.

It is to be noted that various elements of any one embodiment of the invention may be used to replace functionally analogous components in other embodiments. For instance, any one of capacitive pressure sensors 20, 207, 612 and 906 (FIGS. 2, 20, 31 and 37, respectively) may be implemented in a particular case by multilayer ceramic capacitor 224 (FIG. 23) or electrolyte-containing capacitor 510, 540, 550 (FIGS. 28-30) or MEMS capacitor 702 (FIGS. 43-46). The circuit components and current paths of any one embodiment for connecting the pressure measuring circuit inside a patient to detection components and ancillary electrical circuitry outside the patient may be replaced by like-purpose components and paths from other embodiments, e.g., the intravascular circuit path of FIG. 31 (as well as the variations thereof shown in FIGS. 32-36) including core wire 601 and electrodes 603, 604 may be used with resonator 206 and capacitor 207 of FIG. 20 or the ceramic sensor 210 of FIGS. 21 and 22. The processing systems 238 and 266 of FIGS. 24 and 25 may be used in any pressure measuring resonant circuit disclosed herein.

As depicted in FIGS. 47A and 47B, an in vivo pressure sensing system comprises a FFR catheter 1998 having a sensor 1906 at its distal end portion. The FFR catheter 1998 may be inserted in the body of a patient over a standard guide wire 1900. The FFR catheter 1998 is a small flexible device that is introduced over a guide wire 1100 inserted in a patient's body P through blood vessels and vascular structures, such as to the site of a damaged or diseased blood vessel, as typically performed in interventional cardiology. A detection unit 1908 is connected to a ground electrode 1904 attached to the patient's body P and to the proximal end of the FFR catheter 1998 through a contact clip 1996. The distal end of the FFR catheter is connected to the patient's bloodstream through an electrode 1902 exemplarily in the form of a ring at the distal catheter section or a metalized catheter tip. Impedance and/or phase information from the sensor 1906 is extracted by detection unit 1908 through the human body (soft or hard tissue) and contact clip 1996 on the FFR catheter shaft. The guide wire 1900 may be typical of a guide wire used in interventional cardiology or interventional radiology (i.e., composed of non-corrosive biocompatible material(s)) and of a diameter and sufficiently flexibly and bendable to pass through blood vessel(s) or vascular structure(s) to the site to be operated upon in the patient's body. One of the advantages of using an FFR catheter is that the operator can utilize a standard or off-the-shelf guide wire of his choice. The pressure sensitive capacitive element 1906 is a capacitor with at least one pressure sensitive membrane which varies the capacitance responsive to the amount of pressure applied onto the membrane. These pressure sensitive capacitors are well known and described for example in Journal of Micromechanics and Micro-engineering, Volume 17, July 2007; A fast telemetric pressure and temperature sensor system for medical applications; R Schlierf, U Horst, M Ruhl, T Schmitz-Rode, W Mokwa and U Schnakenberg. However, due to the size restrictions inside a FFR catheter the sensors must be no larger than about 200 microns×200 microns×1 mm. Such small sensors as referenced above are typically based on membranes separated by air or vacuum and do not provide enough capacitive change over the physiological blood pressure range to be detected through ground (patient body) impedance and catheter/body parallel capacitance. The typical change in capacitance obtainable with the above referenced type of pressure sensor is in the 10% range which equates to 1 pF or less given a base capacitance of 10 pF or less. Such small capacitive changes cannot be detected directly without amplifying the signal first at the sensor site because a leakage capacitance of 100 pF or higher between FFR catheter and surrounding blood exists. A much higher capacitive change in the order of 100% and a base capacitance of around 1000 pF is desirable in order to enable direct sensing through ground electrode 1904 and catheter shaft 1998 via clip contact 1996.

In another embodiment, instead of using a ground electrode on the patient's skin a ground electrode is mounted on the distal sheath end 1994 making connection with the patient's bloodstream as described hereinabove. Yet another embodiment is to utilize the guide-wire 1900 as the ground or return path. In this embodiment the metalized catheter tip 1902, which forms a distal catheter ground electrode, is replaced with a brush contact (not shown) in the lumen of the distal end of the catheter 1998. Instead of connecting the system 1908 to a ground electrode 1904 the system connects to the proximal guide-wire end through a second electrical clip (not shown).

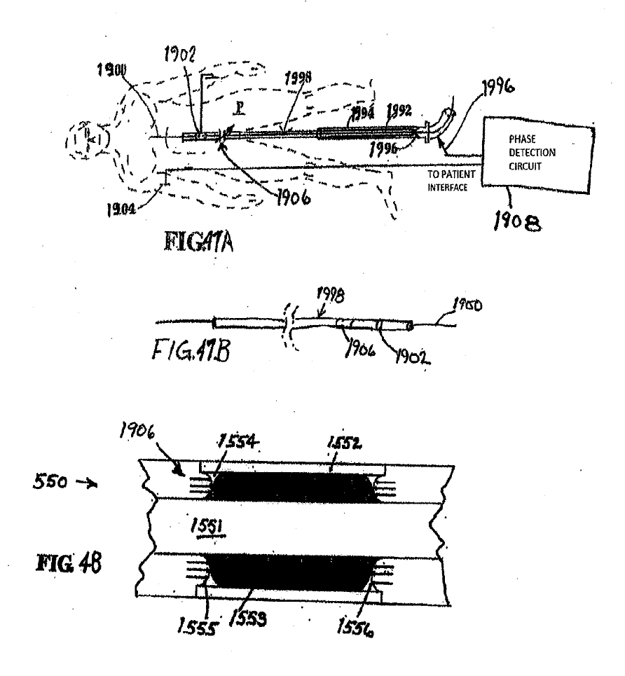

In a particular embodiment of the FFR catheter 1998 of FIGS. 47A and 47B, FIG. 48 shows a distal portion 1550 of the catheter in more detail. In this embodiment the sensor 1906 comprises a pressure sensitive cylindrical element (not separately designated) filled with electrolyte 1553 and mounted in the catheter to detect the blood pressure surrounding the catheter 1998. A lumen 1551 of the catheter forms a path for receiving guide wire 1900 and may be provided with a metal coating or layer in the area of sensor 1906, which serves as an inner electrode. Membranes 1555 and 1556 are variably deformed in accordance with the magnitude of ambient blood pressure, thereby varying the electrolyte volume 1553 in inverse relationship to the combined volume of one or more air pockets 1554 to change the area of contact between the electrolyte 1553 and an outer tubular or cylindrical electrode 1552. (Membranes 1555 and 1556 are exposed to or in contact with ambient fluid, e.g., in chambers flanking sensor 1906 within catheter distal end portion 1550.) Electrode 1552, formed as a metal layer or coating along an outer catheter surface, does not change its configuration in response to change in ambient pressure. This OTW configuration allows to keep the overall catheter diameter to a minimum and therewith reduce the flow impact of the measurement catheter and therewith increase the accuracy of the FFR measurement. The droplet capacitor as described in the journal article “Droplet-based interfacial capacitive sensing” Lab Chip, 2012, v. 12, p. 110-1118: Boaqing Nie et al (copy appended hereto as Exhibit B) would offer the high base capacitance and desired sensitivity. FIG. 48 describes a way to mount such droplet capacitor into an OTW FFR sensing catheter. A typical pressure sensitive capacitor such as such as described for example in Sensors and Actuators A: Physical Vol 73, Issues 1-2, 9 Mar. 1999, Pages 58-67, cannot be used since the size restrictions will limit the capacitance to several pF. Such small capacitance cannot be directly sensed, as proposed herein, through the patient body and the catheter shaft. Different embodiments for circular electrolyte capacitive sensors which can be integrated into the distal catheter portion 1550 are described hereinabove.

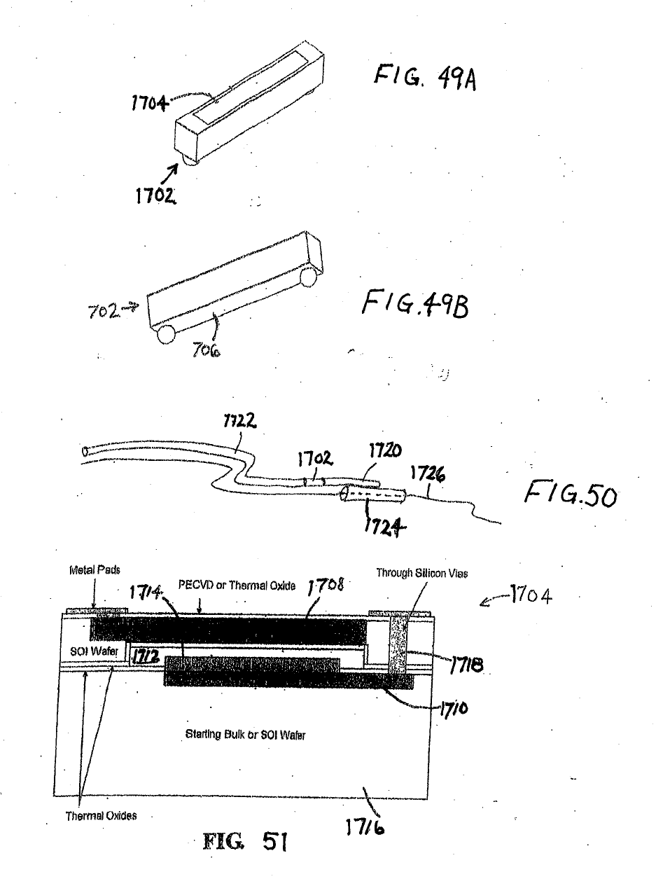

Shown in FIGS. 49A, 49B and 51 is another embodiment of a capacitive sensor 1702 as used in the present invention. Sensor 1702 may be disposed proximate a distal end portion 1720 of an elongate flexible member 1722 as shown in FIG. 50. Elongate flexible member 1722 may take the form of a catheter with a metalized inner surface or the form of a solid metal wire. Distal end portion 1720 of elongate flexible member 1722 is attached to an outer surface of a sleeve 1724 which serves to slidably couple elongate flexible member 1722 at its distal end to a guide wire 1726: guide wire 1726 keeps the FFR catheter positioned inside the blood vessel through sleeve 1724. The sensor 1702 maybe manufactured using semiconductor techniques and may take the form of a MEMS capacitive pressure sensor with a size of 0.2×0.2×1.2 mm (width×depth×length) or less however, providing a capacitance of 0.5-5 nF.

As shown in FIG. 51, MEMS pressure sensor 1702 may be constructed using two silicon wafers 1704 and 1716 that are micro machined, stacked and bonded together. In the fabrication process, SOI (silicon on insulator) wafers may be used to precisely control etching steps and provide robust handling means during fabrication. Metal pads on one side of the wafer may be used for solder, wire bonds or other form of electrical interconnection to the catheter shaft and the distal ground electrode. Such a MEMS type capacitive sensor is designed to achieve 0.5 to 5.0 nF total capacitance. Two plates 1710 and 1708 are separated by a specified gap. The bottom plate 1710 is held fixed while the other plate 1708 deflects with applied pressure. In between the two plates is a vacuum gap 1712 and a dielectric 1714 with a high dielectric constant as for example a PZT material. As pressure is applied to the top plate 1708 it will deflect through the vacuum gap 1712 until it contacts the dielectric material 1714. Once the top plate makes contact with the dielectric the capacitor turns on. As pressure is increased, the area of contact of top plate and dielectric increases which increases the capacitance. The purpose of the dielectric material is to significantly increase the capacitance achievable between top and bottom electrodes. The capacitance prior to top plate 1708/dielectric 1714 contact is negligible. The minimum pressure range of the device is specified by a minimum area of contact between the top plate and the dielectric. The maximum capacitance is defined when a saturation pressure is reached and maximum area of contact is achieved. As the area of contact changes between the top plate 1708 and the dielectric 1714, the capacitance changes and this change in capacitance is proportional to the applied pressure. The high level of capacitance is needed to ensure the electrical signal can be channeled outside of the body without prior amplification while maintaining a high signal to noise ratio

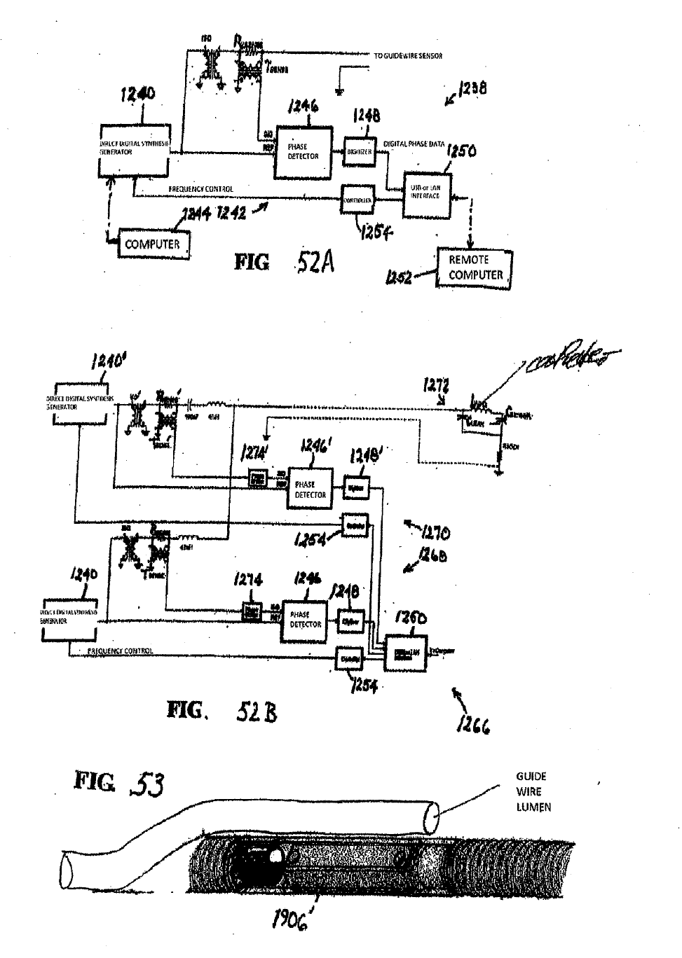

FIG. 52A is a block diagram of a phase detection system 1238 usable as detection circuit 1908, with any suitable sensor arrangement such as those discussed hereinabove with reference to FIGS. 47A, 47B, 48, 49A, 49B, 50, 51, whether in an OTW catheter assembly or in a rapid exchange version with an elongate flexible member 1722 (FIG. 50) (compare U.S. Pat. No. 8,485,985). The electronic circuit in the external system 1238 acts like a network impedance analyzer. It measures amplitude and phase of the whole catheter assembly inserted inside the patient. Phase shift of the AC current into the catheter versus the applied voltage is measured. The system comprises an electronic signal processing circuit configured to monitor electrical current phase changes. The signal processing circuit preferably includes an oscillator 1240, a current sensor connected to a phase detector 1246, a digitizer 1248 and an interface 1250 operatively connected to a computer 1252. An alternative electronic processing system 1266 shown in FIG. 52B and utilizable in an OTW catheter assembly or in a rapid exchange version with an elongate flexible member 1722 compensates for changes in leakage capacitance 1272 that occur naturally between a coated catheter shaft and the blood and body of a patient. These leakage capacitances 1272 change when the catheter is moved forth and back. A first section 1268 of the processing system 1266 is, as in embodiment of FIG. 52A, the phase detection unit that senses pressure. Phase detection circuit 1268 has components that are identical to those in FIG. 52A and bearing the same reference designations. Processing section 1268 is operated at a frequency much lower than that of a second processing section 1270 and is designed to be insensitive to frequencies used in the second section 1270. Catheter movements will cause changes in the leakage capacitance C_{LEAK} 1272 between catheter shaft and patient body, resulting in false pressure change indications. Filters to set the spectral sensitivities of the 2 sections have been omitted for clarity and are easy to design by one skilled in the art, since essentially they are just LC filters. Phase shifters 1274 and 1274′ are needed because most ordinary phase detection circuits suffer from ambiguity every 180 degrees and also from inaccuracy when operated too close to 180 degrees in phase shift or multiples thereof.

The second or upper circuit section 1270 in FIG. 52B has components that are analogous to respective components of the circuit of FIG. 52B and bear the same reference designations with a prime mark. Because of the inductance L_{CATHETER} of the catheter shaft the second circuit section 1270 will indicate the leakage capacitance C_{LEAK} but not the pressure measuring capacitance C_{SENSOR}. Therefore the phase information gathered in the upper or circuit section 1270 will indicate the leakage capacitance C_{LEAK} 1272. This information can be used in the system software to compensate for the amount of false pressure change information caused by a change in the leakage capacitance C_{LEAK} 1272 and greatly improves the pressure reading accuracy of the system in a clinical setting, where wire movements are part of the routine procedure.

FIG. 52 shows a MEMS sensor 1906 integrated into an rapid exchange FFR catheter. Since the catheter shaft does not need to contain signal communication lines like electrical wires or an optical fiber it can be constructed out of solid metal and provide the necessary mechanical performance like torque-ability and push-ability with a minimal diameter. This is important since the catheter shaft crosses the lesion and therewith impacts the blood flow across the lesion. Given the minimal catheter shaft diameter in this invention, which is in the order of a guide wire diameter, this flow impact can be ignored.

1. A system for detection of a physiological parameter, comprising: an elongate flexible member insertable into a subject; a sensor element mounted to a distal end portion of said elongate flexible member; a first electrode at or proximate said distal end portion of said elongate flexible member, said first electrode being disposable in electrically conductive contact with an internal fluid of a subject; a second electrode disposable in contact with a surface of the subject; and a detector circuit conductively coupled to said sensor via a circuit including said first electrode, said elongate flexible member, and said second electrode, said detector circuit being configured to determine an electrical signal parameter indicative of fluid pressure about said distal end portion of said elongate flexible member.

2. A system according to claim 1, wherein said system includes a guide wire and said elongate flexible member extends alongside, outside of, and parallel to said guide wire; said sensor is a capacitive sensor; said first electrode is disposed distally of said capacitive sensor, said capacitive sensor being electrically connectable through said first electrode with the internal fluid inside the subject; said system further includes a contact or a clip at a proximal end of said elongate flexible member, said elongate flexible member serving in part to close said detector circuit through said contact or clip; and said detector circuit includes said contact or clip.

3. A system according to claim 2 wherein said electrical signal parameter is phase shift.

4. A system according to claim 3 wherein said elongate flexible member is a solid metal shaft or wire, said elongate flexible member being slidably coupled to said guide wire via a sleeve connected to said distal end portion of said elongate flexible member, said guide wire passing through said sleeve.

5. A system according to claim 4 wherein said solid metal shaft or wire has a width or diameter of 0.0014″ or less, to minimize flow impact.

6. The system according to claim 2 wherein said detector circuit is configured for monitoring electrical current phase changes from said sensor which vary responsive to the amount of pressure from fluid surrounding said distal end portion of said elongate flexible member.

7. The system according to claim 6 wherein said detector circuit is configured for executing a network analysis method to determine capacitance of said sensor.

8. The system according to claim 6 wherein said detector circuit is configured for executing a complex Fast Fourier Transformation or a single algorithm to determine capacitance of said capacitive sensor.

9. The system according to claim 6 wherein detector circuit includes an oscillator, a current sensor, a phase detector, a digitizer and an interface, said interface being operatively connected to a computer device.

10. The system according to claim 9 wherein said oscillator is a direct digital synthesis generator.

11. The system according to claim 6 wherein said detector circuit includes a first circuit for compensating for measurement error arising from leakage capacitance changes due to varying catheter position, said detector circuit further including a second circuit for detecting capacitance changes of said capacitive sensor caused by fluid pressure changes.

12. The system according to claim 11 wherein said first circuit is configured for operating in a first frequency range and said second circuit is configured for operating in a second frequency range, said second frequency range being much lower than said first frequency range, said second circuit being configured to be insensitive to frequencies in said first frequency range.

13. The system as claimed in claim 1 wherein said elongate flexible member is a tubular catheter, said sensor taking the form of a pressure sensitive electrolyte capacitor.

14. The system according to claim 13 wherein said electrolyte capacitor has an inner electrode inside a lumen of said catheter and an outer electrode on an outer side of said catheter.

15. The system according to claim 14 wherein said sensor includes a membrane that is variously deformable in accordance with external or ambient pressure to vary an electrolyte/electrode contact area inside said sensor.

16. The system according to claim 1 wherein said sensor is electrically connected to a proximal portion of said elongate flexible member and to an electrically conductive distal end portion of said elongate flexible member.

17. The system according to claim 1 wherein said elongate flexible member serves a double purpose as a mechanical support member and an electrical connection to a clip contact outside the subject.

18. The system according to claim 1 wherein said sensor is a semiconductor sensor with ferroelectric or other dielectric material with at least one pressure variable electrode contact area.

19. The system according to claim 1 wherein said sensor comprises two capacitors in parallel, at least one of said capacitors including two plates in the form of ion implanted electrodes separated by an air gap and a dielectric with a high dielectric constant, a first plate of said plates being essentially rigid and in firm contact with the dielectric material, a second plate being flexible so as to deflect under applied pressure to contact said dielectric material with an area of contact varying in accordance with applied pressure.

20. A system according to claim 1, wherein said elongate flexible member is taken from the group consisting of a guide wire, a catheter and a member inserted outside of an parallel to a guide wire or catheter.

21. A method of measuring fluid pressure, comprising: inserting the distal portion of an elongate catheter over a standard guide-wire into fluid at a predetermined site, said distal portion being provided with a capacitive pressure sensor; detecting a phase shift current signal from the capacitive sensor, and determining a fluid pressure value from the detected phase shift.

22. A system for detection of a physiological parameter, comprising: a catheter having a metal catheter shaft, a capacitive sensor at a distal portion of the catheter, a first electrode distal to the sensor configured for making electrical contact with ambient fluid, a second contact made through a clip on the proximal portion of said catheter shaft.

23. The system of claim 22 wherein said ambient fluid is blood of a patient.

24. The system of claim 22 wherein said clip contact on the proximal wire portion is connected to a phase detection circuit with a second electrode electrically connected to the ambient fluid surrounding the catheter.

25. The system of claim 22 wherein said catheter has a distal and a proximal end, said capacitive sensor is disposed at said distal end of said catheter; said first electrode being a ground electrode, said capacitive sensor being electrically connectable through said ground electrode of said catheter with the ambient fluid, said capacitive sensor being conductively connected to said metal shaft of said catheter, and a clip being provided at said proximal end of said catheter shaft to close an electrical circuit through the clip.

26. The system according to claim 25 wherein said distal end of said catheter has at least a portion coated with an electrically conductive medium constituting said ground electrode to connect the circuit with the ambient fluid.

27. The system according to claim 25 wherein said capacitive sensor is taken from the group consisting of a liquid electrolyte filled capacitive sensor and a semiconductor sensor with ferroelectric or other dielectric material with pressure variable electrode contact area.

28. The system according to claim 23 wherein an electrical ground connection is made through a guide-wire, a brush contact in a distal inner catheter section and a second contact clip attached to a proximal guide-wire end.

29. The system according to claim 25 wherein said catheter has a lumen traversed by a guide wire having a distal end and a proximal end, said catheter having a distal end portion; said first electrode being disposed distally of said capacitive sensor, the system including a second electrode disposable in contact with a surface of a patient, a sheath or guide catheter contact or clip being provided at a proximal end of said catheter, a detector circuit being conductively coupled to said capacitive sensor via a circuit including said first electrode, said catheter, said contact or clip, and said second electrode.

30. A system for detection of a physiological parameter comprising:
a guide wire having a distal end and a proximal end;
an elongate flexible member extending outside said guide wire and parallel thereto, said elongate flexible member having a distal end portion coupled to said guide wire;
a capacitive sensor disposed within said distal end portion of said elongate flexible member;
a first electrode conductively connected to said capacitive sensor and disposed distally of said capacitive sensor, said capacitive sensor being electrically connectable through said first electrode with the blood stream of a patient;
a sheath or guide catheter contact or a clip at a proximal end of said elongate flexible member, said elongate flexible member serving in part to close an electrical circuit through said contact or clip;
a second electrode disposable in contact with a surface of a patient; and
a detector circuit conductively coupled to said capacitive sensor via a circuit including said first electrode, said elongate flexible member, said contact or clip, and said second electrode.
